(19) United States
(12) Patent Application Publication
(10) Pub. No.: US 2006/0223834 A1
(43) Pub. Date: Oct. 5, 2006

(54) Diketo acids on nucleobase scaffolds as inhibitors of Flaviviridae

(76) Inventors: Vasu Nair, Athens, GA (US); Guochen Chi, Athens, GA (US); Vinod R. Uchil, Athens, GA (US)

Correspondence Address: Henry D. Coleman, 714 Colorado Avenue, Bridgeport, CT, 06605, US

(21) Appl. No.: 11/098,125

(22) Filed: Apr. 4, 2005

(51) Int. Cl. A61K 31/522; A61K 31/513

(52) U.S. Cl. 514/263.3; 514/263.36; 514/269

(57) ABSTRACT

A new class of diketo acids constructed on nucleobase scaffolds, designed as inhibitors of HCV replication through inhibition of HCV NS5B RNA polymerase, is described. These compounds are useful in the prevention or treatment of infection by HCV and in the treatment of other Flaviviridae infections, either as the compounds, or as pharmaceutically acceptable salts, with pharmaceutically acceptable carriers, used alone or in combination with antivirals, immunomodulators, antibiotics, vaccines, and other therapeutic agents. Methods of treating HCV and methods of treating or preventing infection by HCV are also described.

### FIELD OF THE INVENTION

The present invention relates to compounds which are diketoacids of nucleobase scaffolds which are useful for inhibiting viruses of the family Flaviviridae (flaviviruses), for treating or reducing the likelihood of infections which have a virus from the family Flaviviridae as the causative agent.

### BACKGROUND OF THE INVENTION

Viruses of the family, Flaviviridae, are positive, single-stranded RNA viruses that include some well-known viruses and others that are not so well known and perhaps others that are as yet unclassified (Fields Virology, Third Edition, 1996, 931-1074). There are three genera within this family: flaviviruses, hepaciviruses and prestiviruses. Flaviviruses include those of the Dengue group (Types 1-4), the Japanese Encephalitis group (including the West Nile virus), the Modoc virus group, the Rio Bravo virus group, the Ntaya virus group, the tick-borne encephalitis group, the Tyuleniy virus group, the Uganda virus group and the Yellow Fever virus group. The genus, hepacivirus, of Flaviviridae has only one species, the hepatitis C virus. The third group, pestiviruses, include such viruses as the bovine diarrhea virus, hog cholera virus and border disease virus.

The hepatitis C virus (HCV), identified in 1989 (Choo et al., Science 1989, 244, 359-362), has emerged as a serious global health problem with over 170 million people infected worldwide. A significantly high percentage of those individuals infected with HCV develop chronic liver disease including cirrhosis and hepatocellular carcinoma (Lauer and Walker, N. Engl. J. Med. 2001, 345, 41-52, Lawrence, Adv. Int. Medicine, 2000, 45, 65-105). In addition, many HIV/AIDS patients are also co-infected with HCV (Lawrence, Adv. Int. Medicine, 2000, 45, 65-105). Thus, a major national and global need exists for the discovery and development of therapeutic agents against Flaviviridae infections, and especially against HCV.

HCV is a single-stranded linear RNA virus in the Flaviviridae family (Purcell, Hepatology, 1997, 26, 1IS-14S). The RNA genome has about 9,600 nucleotide units that encode for structural nucleocapsid and envelope proteins and viral enzyme proteins that are necessary for replication. HCV RNA translation produces a large polyprotein which is processed by viral and host enzymes (Major and Feinstone, Hepatology, 1997, 25, 1527-1538). Among the resulting proteins are NS3 RNA protease, NS3 RNA helicase and the NS5B RNA polymerase, all of which have implications for infectivity and response to therapy (Lohmann et al., J. Virol. 1997, 71, 8416-8428; Reed and Rice, Curr. Top. Microbiol. Immunol. 2000, 242, 55-84). Unlike the replication of HBV and HIV, there is no DNA involved in HCV replication. The crystal structure of HCV NS5B polymerase has been reported (Bressanelli, et al, PNAS USA, 1999, 96, 13034-13039).

Perhaps the most effective anti-HCV treatment at present involves combination therapy with ribavarin and alpha-interferon (Lawrence, Adv. Int. Medicine, 2000, 45-65, 105). However, this treatment is effective only for about 40% of patients (Poynard et al., Lancet, 1998, 352, 1426-1432). (See also, PCT Publication Nos. WO 99/59621, WO 00/37110, WO 01/81359, WO 02/32414, WO 03/024461, WO 99/15194, WO 99/64016, and WO 00/24355 for ribavirin and alpha-interferon treatments).

A number of other inhibitors of HCV replication have been discovered and have been suggested as possible treatments for HCV infections, in addition to ribavarin and alpha-interferon (Dymock, Emerging Drugs, 2001, 6, 13-42, Eldrup, et al., J. Med. Chem. 2004, 47, 2283-2295, Dymock et al., Antiviral Chem. Chemother. 2000, 11, 79-96). They include, among others, protease inhibitors, nucleoside analogs and non-nucleoside analogs. Relevant patents for these inhibitors and treatments are: International Publication Nos. WO 98/17679, WO 99/07734, WO 01/32153, WO 01/60315, WO 01/90121, WO 01/92282, WO 02/18404, WO 02/100415, WO 02/32920, WO 2004/013298 A2, WO 02/057425, WO 02/057287, WO 03/026587 A2, WO 2004/032827 A3, WO 2004/043339 A2, WO 2004/111013 A1, WO 2004/009543 A3, U.S. Publications 2003/0050229 A1, 03/0060400 A1, 02/0147160 A1).

However, none of the above cited patents or articles or other related patents or publications (except those cited below) are concerned with diketo acids with potential as anti-HCV agents, which is the subject of our patent application. The inhibitors of direct interest to our patent application are some diketo acids that have been shown recently to be inhibitors of HCV NS5B RNA polymerase (Summa, et al., J. Med. Chem. 2004, 47, 14-17, Altamura, et al. International Publication No. WO 0006529). The most active compounds among this class are shown below.

### IC₅₀ Data for Inhibition of HCV NS5B RNA Polymerase

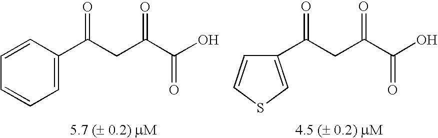

We have designed and synthesized a unique class of diketo acids containing nucleobase scaffolds, that are entirely different from the compounds of the above patents and publications, that are of interest as inhibitors of the replication of viruses of the Flaviviridae family, and especially HCV.

### SUMMARY OF THE INVENTION

A new class of diketo acids constructed on nucleobase scaffolds, and designed as inhibitors of HCV replication through inhibition of HCV NS5B RNA polymerase, is described. These compounds are also of interest as inhibitors of the replication of other viruses of the Flaviviridae family and as antiviral therapeutic agents. The compounds can be represented by the general formula I (and includes tautomers, regioisomers, geometric isomers and optical isomers thereof, as well as pharmaceutically acceptable salts thereof, where applicable), in which the moiety illustrated as a square is a molecular scaffold made up of a nucleic acid base (nucleobase) derivative. These compounds have application in the prevention or treatment of infection by viruses of the Flaviviridae family and especially against HCV, either as the compounds, or as their pharmaceutically acceptable salts, with pharmaceutically acceptable carriers, used alone or in combination with antivirals, immunomodulators, antibiotics, vaccines, and other therapeutic agents.

Pharmaceutical compositions, methods of treating virus infections and related methods of inhibiting HCV NS5B RNA polymerase, as otherwise described herein, are additional aspects of the present invention.

### DETAILED DESCRIPTION OF THE INVENTION

The following terms shall be used throughout the specification to describe the present invention. Unless otherwise indicated, a term used to describe the present invention shall be given its ordinary meaning as understood by those skilled in the art.

The term “compound”, as used herein, unless otherwise indicated, refers to any specific chemical compound disclosed herein and includes tautomers, regioisomers, geometric isomers, and where applicable, optical isomers thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds.

The term “patient” or “subject” is used throughout the specification to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “effective” is used herein, unless otherwise indicated, to describe an amount of a compound or composition which, in context, is used to produce or effect an intended result, whether that result relates to the treatment of a viral, microbial or other disease state, disorder or condition associated with Flaviviridae, especially HCV or alternatively, is used to produce another compound, agent or composition. This term subsumes all other effective amount or effective concentration terms which are otherwise described in the present application.

The term “nucleobase scaffold” is used throughout the specification to mean a nucleoside base selected from uracil, xanthine, hypoxanthine, 8-oxopurine and purine which contain at least four substituents at four substitutable positions on the nucleoside base, one of which is a ketoacid as otherwise defined herein and the other three of which R¹, R² and R³, are as defined herein.

The term “heteroaryl” shall mean a 5 or 6-membered heteroaromatic ring containing 1 to 2 heteroatoms selected from oxygen, nitrogen and sulfur, which heteroaromatic ring is optionally substituted with from 1 to 3 substituents such as halogen, hydroxyl, C₁₋₃ alkyl, C₁₋₃ alkoxy and CF₃. The terms heteroaryl and “heteroaromatic ring” are used interchangeably herein.

The term Flaviviridae is used to describe a family of positive, single-stranded RNA viruses (as used synonymously herein “flaviviruses” that include three genera: flaviviruses, hepaciviruses and prestiviruses which include some well-known viruses and others that are not so well known and perhaps others that are as yet unclassified. Viruses of the family, Flaviviridae, are positive, single-stranded RNA viruses that include some well-known viruses and others that are not so well known and perhaps others that are as yet unclassified. There are three genera within this family: flaviviruses, hepaciviruses and pestiviruses. Flaviviruses include those of the Dengue group (Dengue virus, Dengue virus type 1, Dengue virus type 2, Dengue virus type 3, Dengue virus type 4), the Japanese Encephalitis virus group (Alfuy virus, Japanese encephalitis virus, Kookaburra virus, Koutango virus, Kunjin virus, Murray Valley encephalitis virus, St. Louis encephalitis virus, Stratford virus, Usutu virus, West Nile virus), the Modoc virus, the Rio Bravo virus group (Apoi virus, Rio Bravo virus, Saboya virus), the Ntaya virus, the tick-borne encephalitis group (tick-born encephalitis viruses), the Tyuleniy virus group, the Uganda S virus, and the Yellow Fever virus. The genus, hepacivirus, of Flaviviridae has only one species, the hepatitis C virus (HCV), which is composed of many clades, types and subtypes. The third group, pestiviruses, include the bovine diarrhea virus-2 (BVDV-2), pestivirus type 1 (including BVDV), pestivirus type 2 (including hog cholera virus) and pestivirus type 3 (including border disease virus).

The term “Yellow Fever virus” is used to describe the flavivirus which is the causative agent of yellow fever. Yellow fever is a tropical mosquito-borne viral hepatitis, due to Yellow Fever virus (YFV), with an urban form transmitted by Aedes aegypti, and a rural, jungle or sylvatic form from tree-dwelling mammals by various mosquitos of the Haemagogus species complex. Yellow fever is characterized clinically by fever, slow pulse, albuminuria, jaundice, congesion of the face and hemorrhages, especially hematemesis (black vomit). It is fatal in about 5-10% of the cases.

The term “Dengue virus” is used throughout the specification to describe the flavivirus which is the causative agent(s) of dengue fever/dengue hemorrhagic fever. Dengue is a disease of tropical and subtropical regions occurring epidemically and caused by Dengue virus, one of a group of arboviruses which causes the hemorrhagic fever syndrome. Four grades of severity are recognized: grade I: fever and constitutional symptoms, grade II: grade I plus spontaneous bleeding (of skin, gums or gastrointestinal tract), grade III, grade II plus agitation and circulatory failure and grade IV: profound shock. The disease is transmitted by a mosquito of the genus Aedes (generally A. aegyptii, but frequently, A. albopictus). Also called Aden, bouquet, breakbone, dandy, date, dengue (hemorrhagic) or polka, solar fever, stiffneck fever, scarlatina rheumatica or exanthesis arthorosia. Hemorrhagic dengue is a more pathogenic epidemic form of dengue which has erupted in a number of epidemic outbreaks in the Pacific region in recent years.

The term HCV refers to hepatitis C viruses of the genus, hepacivirus, which also belong to the family, Flaviviridae.

The term HCV NS5B RNA polymerase refers to the viral enzyme which is a key enzyme for the replication of HCV.

The disease known as AIDS (acquired immunodeficiency syndrome) caused by the human immunodeficiency virus (HIV) is often accompanied by HCV infection in AIDS patients with resulting serious additional health consequences arising from this HCV co-infection. Thus, coadministration of anti-HCV, anti-HIV and other drugs may be a necessary regimen for treatment of such patients. “ARC” and “AIDS” which refer to syndromes of the immune system caused by HIV and are characterized by susceptibility to certain diseases and T cell counts which are depressed compared to normal counts. HIV progresses from Category 1 (Asymptomatic HIV Disease) to Category 2 (ARC), to Category 3 (AIDS), with the severity of the disease.

A Category 1 HIV infection is characterized by the patient or subject being HIV positive, asymptomatic (no symptoms) and having never had fewer than 500 CD4 cells. If the patient has had any of the AIDS-defining diseases listed for categories 2 (ARC) or 3 (AIDS), then the patient is not in this category. If the patient's t-cell count has ever dropped below 500, that patient is considered either Category 2 (ARC) or Category 3 (AIDS).

A Category 2 (ARC) infection is characterized by the following criteria: The patient's T-cells have dropped below 500 but never below 200, and that patient has never had any Category 3 diseases (as set forth below) but have had at least one of the following defining illnesses:
Bacillary angiomatosis
Candidiasis, oropharyngeal (thrush)
Candidiasis, vulvovaginal; persistent, frequent, or poorly responsive to therapy
Cervical dysplasia (moderate or severe)/cervical carcinoma in situ
Constitutional symptoms, such as fever (38.5 C) or diarrhea lasting longer than 1 month
Hairy leukoplakia, oral
Herpes zoster (shingles), involving at least two distinct episodes or more than one dermatome
Idiopathic thrombocytopenic purpura
Listeriosis
Pelvic inflammatory disease, particularly if complicated by tubo-ovarian abscess
Peripheral neuropathy.

According to the U.S. government, in. Category 2 ARC, the immune system shows some signs of damage but it isn't life-threatening.

A Category 3 (AIDS) infection is characterized by the following criteria:
your T-cells have dropped below 200 or
you have had at least one of the following defining illnesses:
Candidiasis of bronchi, trachea, or lungs
Candidiasis, esophageal
Cervical cancer, invasive**
Coccidioidomycosis, disseminated or extrapulmonary
Cryptococcosis, extrapulmonary
Cryptosporidiosis, chronic intestinal (greater than 1 month's duration)
Cytomegalovirus disease (other than liver, spleen, or nodes)
Cytomegalovirus retinitis (with loss of vision)
Encephalopathy, HIV-related
Herpes simplex: chronic ulcer(s) (greater than 1 month's duration); or bronchitis, pneumonitis, or esophagitis
Histoplasmosis, disseminated or extrapulmonary
Isosporiasis, chronic intestinal (greater than 1 month's duration)
Kaposi's sarcoma
Lymphoma, Burkitt's (or equivalent term)
Lymphoma, immunoblastic (or equivalent term)
Lymphoma, primary, of brain
Mycobacterium avium complex or M. kansasii, disseminated or extrapulmonary
Mycobacterium tuberculosis, any site (pulmonary** or extrapulmonary)
Mycobacterium, other species or unidentified species, disseminated or extrapulmonary
Pneumocystis carinii pneumonia
Pneumonia, recurrent**
Progressive multifocal leukoencephalopathy
Salmonella septicemia, recurrent
Toxoplasmosis of brain
Wasting syndrome due to HIV.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time.

The present invention is directed to compounds of the general molecular formula I, combinations thereof, or pharmaceutically acceptable salts thereof, in the inhibition of HCV NS5B RNA polymerase, the prevention or treatment of HCV infections and in the treatment of hepatitis C. These compounds are also of interest in the prevention or treatment of infections caused by other viruses of the Flaviviridae family. Compounds of formula I are defined as follows: 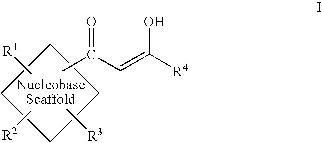
including tautomers, regioisomers, geometric isomers, and where applicable, optical isomers thereof, and pharmaceutically acceptable salts thereof, wherein the nucleobase scaffold and R groups are defined as:

(i) keto acids with uracil nucleobase scaffold; 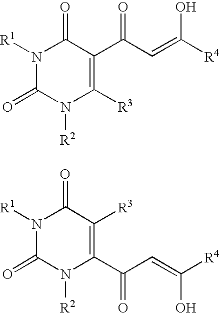
R¹ and R² are independently:
   a) H,
   b) C₁₋₆ alkyl,
   c) C₁₋₆ fluoroalkyl,
   d) C₁₋₆ alkyl. S(O)ₙR, wherein n selected from 0-2, R is selected from C₁₋₃ alkyl, phenyl and substituted phenyl with substituents selected from:
      1) halogen,
      2) hydroxy,
      3) C₁₋₃ alkyl,
      4) C₁₋₃ alkoxy,
      5) CF₃,
   e) C₅₋₆ cycloalkyl with 1 to 3 substituents selected from:
      1) halogen,
      2) hydroxy,
      3) C₁₋₃ alkyl,
      4) C₁₋₃ alkoxy,
      5) CF₃,
   f) C₁₋₆ alkenyl,
   g) C₁₋₆ alkyl COₙR^{a}, wherein n selected from 1 and 2, R^{a} selected from:
      1) C₁₋₆ alkyl,
      2) H,
   h) Phenyl,
   i) Substituted phenyl with 1 to 3 substituents selected from:
      1) halogen,
      2) hydroxy,
      3) C₁₋₃ alkyl,
      4) C₁₋₃ alkoxy,
      5) CF₃,
   j) Benzyl,
   k) Substituted benzyl with 1 to 3 substituents selected from:
      1) halogen,
      2) hydroxy,
      3) C₁₋₃ alkyl,
      4) C₁₋₃ alkoxy,
      5) CF₃,
   l) C₂₋₆ alkyl substituted with phenyl,
   m) C₂₋₆ alkyl substituted with phenyl, the phenyl group may be substituted with 1 to 3 substituents selected from:
      1) halogen,
      2) hydroxy,
      3) C₁₋₃ alkyl,
      4) C₁₋₃ alkoxy,
      5) CF₃,
   n) R^{b},
   o) C₁₋₆ alkyl substituted with R^{b},

Wherein each R^{b} is 5 or 6 membered heteroaromatic ring containing 1 to 2 heteroatoms selected from oxygen, nitrogen and sulfur, the ring could be substituted or not on carbon or nitrogen with 1 to 3 substituents selected from:
1) halogen,
2) hydroxy,
3) C₁₋₃ alkyl,
4) C₁₋₃ alkoxy,
5) CF₃,
R³ is selected from:
   a) H,
   b) C₁₋₆ alkyl,
   c) Halogen,
   d) Hydroxyl,
   e) Phenylthio,
   f) Substituted phenylthio with 1 to 3 substituents selected from:
      1) halogen,
      2) hydroxy,
      3) C₁₋₃ alkyl,
      4) C₁₋₃ alkoxy,
      5) CF₃,
   g) Benzyl,
   h) Substituted benzyl with 1-3 substituents selected from:
      1) halogen,
      2) hydroxy,
      3) C₁₋₃ alkyl,
      4) C₁₋₃ alkoxy,
      5) CF₃,
R⁴ is selected from:
   CO₂R^{c}, wherein R^{c} is selected from:
      1) C₁₋₆ alkyl,
      2) H,
      3) sodium or other pharmaceutical acceptable salt,

(ii) keto acids with xanthine nucleobase scaffold; 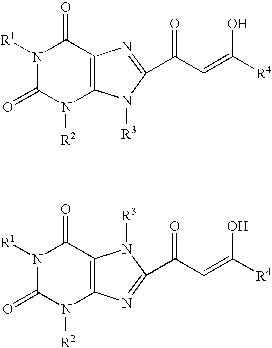
R¹, R² and R³ are independently:
   a) H,
   b) C₁₋₆ alkyl,
   c) C₁₋₆ fluoroalkyl,
   d) C₁₋₆ alkyl S(O)ₙR, wherein n selected from 0-2, R selected from C₁₋₃ alkyl, phenyl and substituted phenyl with substituents selected from:
      1) halogen,
      2) hydroxy,
      3) C₁₋₃ alkyl,
      4) C₁₋₃ alkoxy,
      5) CF₃,
   e) C₅₋₆ cycloalkyl with 1 to 3 substituents selected from:
      1) halogen,
      2) hydroxy,
      3) C₁₋₃ alkyl,
      4) C₁₋₃ alkoxy,
      5) CF₃,
   f) C₁₋₆ alkenyl,
   g) C₁₋₆ alkyl COₙR^{a}, wherein n selected from 1 and 2, R^{a} selected from:
      1) C₁₋₆ alkyl,
      2) H,
   h) Phenyl,
   i) Substituted phenyl with 1 to 3 substituents selected from:
      1) halogen,
      2) hydroxy,
      3) C₁₋₃ alkyl,
      4) C₁₋₃ alkoxy,
      5) CF₃,
   j) Benzyl,
   k) Substituted benzyl with 1 to 3 substituents selected from:
      1) halogen,
      2) hydroxy,
      3) C₁₋₃ alkyl,
      4) C₁₋₃ alkoxy,
      5) CF₃,
   l) C₂₋₆ alkyl substituted with phenyl,
   m) C₂₋₆ alkyl substituted with phenyl, the phenyl group may be substituted with 1 to 3 substituents selected from:
      1) halogen,
      2) hydroxy,
      3) C₁₋₃ alkyl,
      4) C₁₋₃ alkoxy,
      5) CF₃,
   n) R^{b},
   o) C₁₋₆ alkyl substituted with R^{b},

Wherein each R^{b} is 5 or 6 membered heteroaromatic ring containing 1 to 2 heteroatoms selected from oxygen, nitrogen and sulfur, the ring could be substituted or not on carbon or nitrogen with 1 to 3 substituents selected from:
1) halogen,
2) hydroxy,
3) C₁₋₃ alkyl,
4) C₁₋₃ alkoxy,
5) CF₃,

R⁴ is selected from:
CO₂R^{c}, wherein R^{c} is selected from:
   1) C₁₋₆ alkyl,
   2) H,
   3) sodium or other pharmaceutical acceptable salt.

(iii) keto acids with hypoxanthine and 8-oxopurine nucleobase scaffolds;

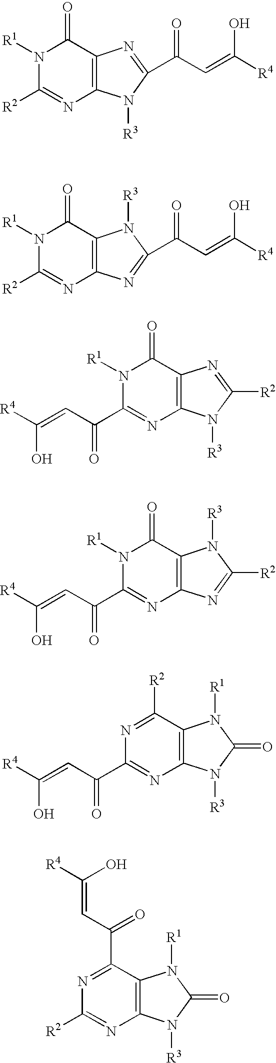

R¹, R² and R³ are independently:
a) H,
b) C₁₋₆alkyl,
c) C₁₋₆ fluoroalkyl,
d) C₁₋₆ alkyl S(O)ₙR, wherein n selected from 0-2, R selected from C₁₋₃ alkyl, phenyl and substituted phenyl with substituents selected from:
   1) halogen,
   2) hydroxy,
   3) C₁₋₃ alkyl,
   4) C₁₋₃ alkoxy,
   5) CF₃,
e) C₅₋₆cycloalkyl with 1 to 3 substituents selected from:
   1) halogen,
   2) hydroxy,
   3) C₁₋₃ alkyl,
   4) C₁₋₃ alkoxy,
   5) CF₃,
f) C₁₋₆ alkenyl,
g) C₁₋₆ alkyl COₙR^{a}, wherein n selected from 1 and 2, R^{a} selected from:
   1) C₁₋₆ alkyl,
   2) H,
h) Phenyl,
i) Substituted phenyl with 1 to 3 substituents selected from:
   1) halogen,
   2) hydroxy,
   3) C₁₋₃ alkyl,
   4) C₁₋₃ alkoxy,
   5) CF₃,
j) Benzyl,
k) Substituted benzyl with 1 to 3 substituents selected from:
   1) halogen,
   2) hydroxy,
   3) C₁₋₃ alkyl,
   4) C₁₋₃ alkoxy,
   5) CF₃,
l) C₂₋₆ alkyl substituted with phenyl,
m) C₂₋₆ alkyl substituted with phenyl, the phenyl group may be substituted with 1 to 3 substituents selected from:
   1) halogen,
   2) hydroxy,
   3) C₁₋₃ alkyl,
   4) C₁₋₃ alkoxy,
   5) CF₃,
n) R^{b},
o) C₁₋₆ alkyl substituted with R^{b},

Wherein each R^{b} is 5 or 6 membered heteroaromatic ring containing 1 to 2 heteroatoms selected from oxygen, nitrogen and sulfur, the ring could be substituted or not on carbon or nitrogen with 1 to 3 substituents selected from:
1) halogen,
2) hydroxy,
3) C₁₋₃ alkyl,
4) C₁₋₃ alkoxy,
5) CF₃,

R⁴ is selected from:
CO₂R^{c}, wherein R^{c} is selected from:
   1) C₁₋₆ alkyl,
   2) H,
   3) sodium or other pharmaceutical acceptable salt.

(iv) keto acids with purine nucleobase scaffold;

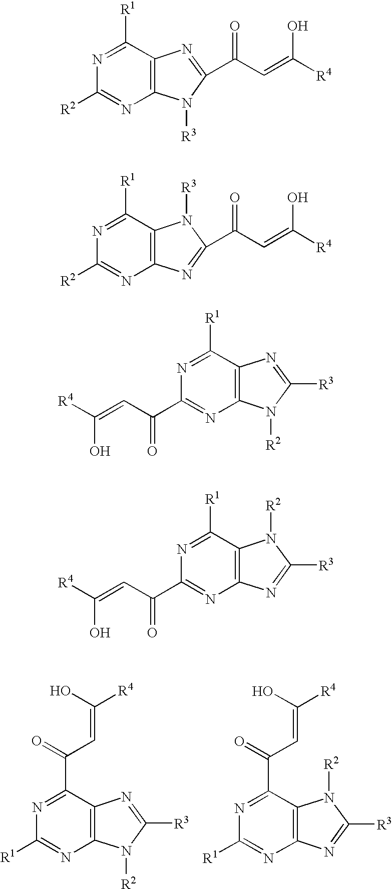

R¹, R² and R³ are independently:
a) H,
b) C₁₋₆ alkyl,
c) C₁₋₆ fluoroalkyl,
d) C₁₋₆ alkyl S(O)ₙR, wherein n selected from 0-2, R selected from C₁₋₃ alkyl, phenyl and substituted phenyl with substituents selected from:
   1) halogen,
   2) hydroxy,
   3) C₁₋₃ alkyl,
   4) C₁₋₃ alkoxy,
   5) CF₃,
e) C₅₋₆ cycloalkyl with 1 to 3 substituents selected from:
   1) halogen,
   2) hydroxy,
   3) C₁₋₃ alkyl,
   4) C₁₋₃ alkoxy,
   5) CF₃,
f) C₁₋₆ alkenyl,
g) C₁₋₆ alkyl COₙR^{a}, wherein n selected from 1 and 2, R^{a} selected from:
   1) C₁₋₆ alkyl,
   2) H,
h) Phenyl,
i) Substituted phenyl with 1 to 3 substituents selected from:
   1) halogen,
   2) hydroxy,
   3) C₁₋₃ alkyl,
   4) C₁₋₃ alkoxy,
   5) CF₃,
j) Benzyl,
k) Substituted benzyl with 1 to 3 substituents selected from:
   1) halogen,
   2) hydroxy,
   3) C₁₋₃ alkyl,
   4) C₁₋₃ alkoxy,
   5) CF₃,
l) C₂₋₆ alkyl substituted with phenyl,
m) C₂₋₆ alkyl substituted with phenyl, the phenyl group may be substituted with 1 to 3 substituents selected from:
   1) halogen,
   2) hydroxy,
   3) C₁₋₃ alkyl,
   4) C₁₋₃ alkoxy,
   5) CF₃,
n) R^{b},
o) C₁₋₆ alkyl substituted with R^{b},

Wherein each R^{b} is 5 or 6 membered heteroaromatic ring containing 1 to 2 heteroatoms selected from oxygen, nitrogen and sulfur, the ring could be substituted or not on carbon or nitrogen with 1 to 3 substituents selected from:
1) halogen,
2) hydroxy,
3) C₁₋₃ alkyl,
4) C₁₋₃ alkoxy,
5) CF₃,

R⁴ is selected from:
CO₂R^{c}, wherein R^{c} is selected from:
   4) C₁₋₆ alkyl,
   5) H,
   6) sodium or other pharmaceutical acceptable salt.

Also included within the present invention are pharmaceutical compositions useful for inhibiting HCV NS5B RNA polymerase, comprising of an effective amount of a compound of this invention, and a pharmaceutically acceptable carrier. Pharmaceutical compositions useful for treating infection by HCV or for treating hepatitis C or other flaviviruses are also included by the present invention. The present invention also includes methods for inhibiting the viral enzyme, HCV NS5B polymerase, and a method of inhibiting HCV growth or replication, or treating an HCV infection. In addition, the present invention has application in the prevention or treatment of infection caused by other viruses of the Flaviviridae family. The present invention is also directed to a pharmaceutical composition comprising, in combination, a therapeutically effective amount of a compound of the present invention in combination with a therapeutically effective amount of an agent selected from: (i) an AIDS or HIV antiviral agent, (ii) an anti-infective agent, (iii) an immunomodulator, (iv) other useful therapeutic agents including antibiotics and other antiviral agents, as otherwise described.

The compounds of the present invention may have regioisomers with respect to R¹, R² and R³ and these regioisomeric forms are included in the present invention. The compounds of the present invention may have asymmetric centers and may occur as optical isomers and all of these isomeric forms are included in the present patent invention. The compounds may have geometric isomers and these forms are included in the present invention.

Tautomeric forms may also exist with compounds of the present invention. Thus, the terminology “and tautomers thereof” is used in describing tautomeric forms of compounds of formula I such as Ia and Ib (shown below). By naming compounds as being represented by the 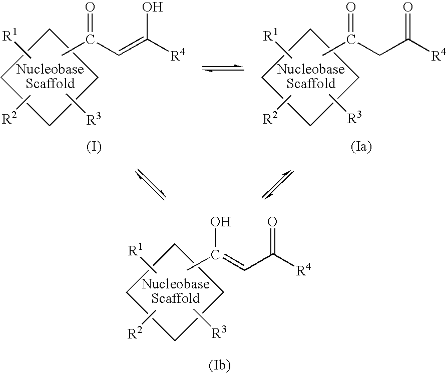
general formula I and tautomers thereof, it is understood that for the purposes of the present invention that tautomers Ia and Ib are also included. Similarly, by referring to compound (Ia), it is understood for the purposes of the present application that the tautomers (I) and (Ib) are also intended. The same holds true for references to tautomer (Ib).

When the variables involving R¹, R², R³, R⁴ occur more than once in any formula I, its definition on each occurrence is independent of its definition at every other occurrence. Combinations of nucleobase and variables are permissible only if such combinations result in stable compounds.

The compounds of the present invention are useful in the inhibition of HCV NS5B RNA polymerase, the prevention or treatment of infection by HCV and in the treatment of the disease known as hepatitis C. Treating hepatitis or preventing or treating infection by HCV is defined as including the treatment of a wide range of states of HCV infection including actual or potential exposure to HCV (e.g., through blood transfusion, exchange of body fluids, bites, needle punctures, exposure to infected patient blood during medical or dental procedures, and other means).

Other applications are also part of this invention. For example, the compounds of this invention are useful in the preparation and execution of screening assays for antiviral compounds including in the isolation of viral enzyme mutants and in further understanding of the enzyme, HCV NS5B RNA polymerase. Applications to other viruses of Flaviviridae are also included in the present application.

The present invention also provides for the use of a compound of structural formula (I) to make a pharmaceutical composition useful for inhibiting HCV NS5B RNA polymerase and in the treatment of HCV infection and hepatitis C.

The compounds of the present invention may be administered in the form of “well-known pharmaceutically acceptable” salts. The latter is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, cainsylate, mucate, carbonate, napsylate, chloride, nitrate, clavulanate, N-methylglucamine, citrate, ammonium salt, dihydrochloride, oleate, edetate, oxalate, edisylate, pamoate, estolate, palmitate, esylate, fumarate, phosphate, diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and others which can be used as a dosage form for modifying the solubility or hydrolysis characteristics or can be used in sustained release or pro-drug formulations. The pharmaceutically acceptable salts of this invention include those with counterions such as sodium, potassium, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide.

Also, in the case of a carboxylic acid (—COOH) or an alcohol group being present, pharmaceutically acceptable esters can be employed, e.g., acetate, maleate, pivaloyloxymethyl and others, more preferably C₁-C₂₀ esters and those esters known in the art for improving solubility or hydrolysis characteristics for use as sustained release or pro-drug formulations.

Therapeutically effective amounts of the compounds of the present invention may be administered to patients orally, parenterally, by inhalation spray, or rectally, in dosage unit formulations containing pharmaceutically-acceptable carriers, adjuvants and vehicles including nanoparticle drug delivery approaches. The term “pharmaceutically acceptable” is meant to infer that the carrier, diluent or excipient must be compatible with the other ingredients of the formulation and not deleterious to the patient or recipient. Pharmaceutical compositions may be in the form of orally-administrable suspensions or tablets, nasal sprays and injectible preparations (injectible aqueous or oleagenous suspensions or suppositories). This method of treatment is part of the invention. The administration approaches used (orally as solution or suspension, immediate release tablets, nasal aerosol or inhalation, injectible solutions or suspensions or rectally administered in the form of suppositories) involve techniques that are well-known in the art of pharmaceutical formulation.

The compounds of this invention can be administered orally to humans in a preferred form (such as tablets) in an effective amount within a preferred dosage range of about 0.1 to 200 mg/kg body weight in divided doses. The specific dose level and frequency of dosage for any particular patient may be varied and will depend upon a variety of factors including compound activity, compound metabolism and duration of action, patient age, body weight, general health, sex, diet, mode and time of administration, rate of excretion, drug combination, the severity of the particular condition, and the condition of the patient undergoing therapy.

The present invention also includes therapeutically effective combinations of the compounds of the present invention, including, for example, anti-HCV agents such as HCV NS5B RNA polymerase inhibitor compounds of formula I with one or more other therapeutic agents such as AIDS antivirals, other antiviral agents, immunomodulators, antiinfectives, antibiotics, vaccines or other therapeutic agents. Some examples are given below.

ANTIVIRAL AGENTS

| Drug Name | Manufacturer | Therapeutic Use |
|---|---|---|
| 097 | Hoechst/Bayer | HIV infection, AIDS, ARC |
| | | (NNRT inhibitor) |
| Amprenivir | Glaxo Wellcome | HIV infection, AIDS, ARC |
| 141W94, GW141 | | (protease inhibitor) |
| Abacavir | Glaxo Wellcome | HIV infection, AIDS, ARC |
| (1592U89) | | (RT inhibitor) |
| GW 1592 | | |
| Acemannan | Carrington Labs | ARC |
| | (Irving, TX) | |
| Acyclovir | Burroughs | HIV infection, AIDS, |
| | Wellcome | ARC, in combination |
| | | with AZT |
| AD-439 | Tanox Biosystems | HIV infection, AIDS, ARC |
| AD-519 | Tanox Biosystems | HIV infection, AIDS, ARC |
| Adefovir | Gilead Sciences | HIV infection ARC, PGL |
| dipivoxil AL-721 | Ethigen | HIV positive, AIDS |
| | (Los Angeles, CA) | |
| Alpha Interferon | Glaxo Wellcome | Kaposi's sarcoma, |
| | | HIV in combination |
| | | w/Retrovir |
| Ansamycin | Adria Laboratories | ARC |
| LM 427 | (Dublin, OH) | |
| | Erbamont | |
| | (Stamford, CT) | |
| Antibody which | Advanced | AIDS, ARC |
| neutralizes pH | Biotherapy | |
| labile alpha | Concepts | |
| aberrant | (Rockville, MD) | |
| Interferon | | |
| AR 177 | Aronex Pharm | HIV infection, AIDS, ARC |
| Beta-fluoro-ddA | National Cancer | AIDS-associated diseases |
| | Institute | |
| BMS-232623 | Bristol-Myers | HIV infection, AIDS, ARC |
| (CGP-73547) | Squibb/Novartis | (protease inhibitor) |
| BMS-234475 | Bristol-Myers | HIV infection, AIDS, ARC |
| (CGP-61755) | Squibb/Novartis | (protease inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening CMV |
| Ganciclovir | | Peripheral CMV Retinitis |
| ddI | Bristol-Myers | HIV infection, AIDS, ARC; |
| Dideoxyinosine | Squibb | combination with AZT/d4T |
| DMP-450 | AVID | HIV infection, AIDS, ARC |
| | (Camden, NJ) | (protease inhibitor) |
| Efavirenz | DuPont Merck | HIV infection, AIDS, ARC |
| (DMP-266) | | (non-nucleoside RT |
| | | inhibitor |
| EL10 | Elan Corp, PLC | HIV infection |
| | (Gainesville, GA) | |
| Famciclovir | Smith Kline | Herpes zoster, herpes |
| | | simplex |
| FTC | Emory University | HIV infection, AIDS, ARC |
| | | (reverse transcriptase |
| | | inhibitor) |
| GS 840 | Gilead | HIV infection, AIDS, ARC |
| | | (reverse transcriptase |
| | | inhibitor) |
| HBY097 | Hoechst Marion | HIV infection, AIDS, ARC |
| | Roussel | (non-nucleoside reverse |
| | | transcriptase inhibitor) |
| Hypericin | VIMRx Pharm. | HIV infection, AIDS, ARC |
| Recombinant | Triton Biosciences | AIDS, Kaposi's sarcoma, |
| Human Interferon | (Almeda, CA) | ARC |
| Beta | | |
| Interferon | Interferon Scienes | ARC, AIDS |
| alfa-n3 | | |
| Indinavir | Merck | HIV infection, AIDS, |
| | | ARC, asymptomatic HIV |
| | | positive; combination |
| | | with AZT/ddI/ddC |
| ISIS-2922 | ISIS | CMV retinitis |
| | Pharmaceuticals | |
| KNI-272 | Natl. Cancer | HIV-associated diseases |
| | Institute | |
| Lamivudine, 3TC | Glaxo Wellcome | HIV infection, AIDS, ARC |
| | | (reverse transcriptase |
| | | inhibitor); also with AZT |
| Lobucavir | Bristol-Myers | CMV infection |
| | Squibb | |
| Nelfinavir | Agouron | HIV infection, AIDS, ARC |
| | Pharmaceuticals | (protease inhibitor) |
| Nevirapine | Boeheringer | HIV infection, AIDS, ARC |
| | Ingleheim | (RT inhibitor) |
| Novapren | Novaferon Labs, | HIV inhibitor |
| | Inc. (Akron, OH) | |
| Peptide T | Peninsula Labs | AIDS |
| Octapeptide | (Belmont, CA) | |
| Sequence | | |
| Trisodium | Astra Pharm. | CVV retinitis, HIV |
| Phosphonoformate | Products, Inc. | infection, other CMV |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. | HIV infection, AIDS, ARC |
| | Tech | |
| | (Houston, TX) | |
| Ritonavir | Abbott | HIV infection, AIDS, ARC |
| | | (protease inhibitor) |
| Saquinavir | Hoffmann-LaRoche | HIV infection, AIDS, ARC |
| | | (protease inhibitor) |
| Stavudine; d4T | Bristol-Myers | HIV infection, AIDS, ARC |
| Didehydrode- | Squibb | |
| oxythymidine | | |
| Valaciclovir | Glaxo Wellcome | Genital HSV & CMV |
| | | infections |
| Virazole | Viratek/ICN | Asymptomatic HIV |
| Ribavirin | (Costa Mesa, CA) | positive, LAS, ARC |
| VX-478 | Vertex | HIV infection, AIDS, ARC |
| Zalcitabine | Hoffmann-LaRoche | HIV infection, AIDS, ARC |
| | | with AZT |
| Zidovudine; AZT | Glaxo Wellcome | HIV infection, AIDS, ARC, |
| | | Kaposi's sarcoma, in |
| | | combination with other |
| | | therapies |
| Tenofovir | Gilead | HIV infection, AIDS, |
| diisoproxil | | (RT inhibitor) |
| fumarate salt | | |
| (Viread ®) | | |
| Combivir ® | GSK | HIV infection, AIDS, |
| | | (RT inhibitor) |
| Abacavir | GSK | HIV infection, AIDS, |
| succinate | | (reverse transcriptase |
| (or Ziagen ®) | | inhibitor) |
| Fuzeon ® | Roche/Trimeris | HIV infection, AIDS, |
| (or T-20) | | viral Fusion inhibitor |

IMMUNO-MODULATORS

| Drug Name | Manufacturer | Therapeutic Use |
|---|---|---|
| AS-101 | Wyeth-Ayerst | AIDS |
| Bropirimine | Pharmacia Upjohn | Advanced AIDS |
| Acemannan | Carrington Labs, | AIDS, ARC |
| | Inc. (Irving, TX) | |
| CL246, 738 | American Cyanamid | AIDS, Kaposi's |
| | Lederle Labs | sarcoma |
| EL10 | Elan Corp, PLC | HIV infection |
| | (Gainesville, GA) | |
| FP-21399 | Fuki Immuno | Blocks HIV fusion with |
| | PHARM | CD4+ cells |
| Gamma Interferon | Genentech | ARC, in combination |
| | | w/TNF |
| Granulocyte | Genetics Institute | AIDS |
| Macrophage Colony | Sandoz | |
| Stimulating Factor | | |
| Granulocyte | Hoeschst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 Interleukin-2 | Cetus | AIDS, in combination |
| | | w/AZT |
| IL-2 Interleukin-2 | Hoffman-LaRoche | AIDS, ARC, HIV, in |
| | Immunex | combination w/AZT |
| IL-2 Interleukin-2 | Chiron | AIDS, increase in CD4 |
| (aldeslukin) | | cell counts |
| Immune Globulin | Cutter Biological | Pediatric AIDS, in |
| Intravenous (human) | (Berkeley, CA) | combination w/AZT |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA | sarcoma, ARC, PGL |
| Imuthiol Diethyl | Merieux Institute | AIDS, ARC |
| Dithio Carbamate | | |
| Alpha-2 Interferon | Schering Plough | Kaposi's sarcoma |
| | | w/AZT, AIDS |
| Methionine- | TNI Pharmaceutical | AIDS, ARC |
| Enkephalin | (Chicago, IL) | |
| MTP-PE Muramyl- | Ciba-Geigy Corp. | Kaposi's sarcoma |
| Tripeptide | | |
| Granulocyte Colony | Amgen | AIDS, in combination |
| Stimulating Factor | | w/AZT |
| Remune | Immune Response | Immunotherapeutic |
| | Corp. | |
| rCD4 Recombinant | Genentech | AIDS, ARC |
| Soluble Human CD4- | | |
| IgG rCD4-IgG | | |
| Hybrids | | |
| Recombinant | Biogen | AIDS, ARC |
| Soluble Human CD4 | | |
| Interferon Alfa 2a | Hoffman-LaRoche | AIDS, ARC Kaposi's |
| | | sarcoma, AIDS, AR, |
| | | combination w/AZT |
| SK&F1-6528 Soluble | Smith Kline | HIV infection |
| T4 | | |
| Thymopentin | Immunobiology | HIV infection |
| | Research Institute | |
| | (Annandale, NJ) | |
| Tumor Necrosis | Genentech | ARC, in combination |
| Factor (TNF) | | w/gamma Interferon |

ANTI-INFECTIVES

| Drug Name | Manufacturer | Therapeutic Use |
|---|---|---|
| Clindamycin with | Pharmacia Upjohn | PCP |
| Primaquine | | |
| Fluconazole | Pfizer | Cryptococcal meningitis, |
| | | candidiasis |
| Pastille Nystatin | Squibb Corp. | prevention of oral |
| Pastille | | candidiasis |
| Ornidyl | Merrell Dow | PCP |
| Eflornithine | | |
| Pentamidine | LyphoMed | PCP treatment |
| Isethionate | (Rosemont, IL) | |
| (IM & IV) | | |
| Trimethoprim | | Antibacterial |
| Trimethoprim/ | | Antibacterial |
| sulfa | | |
| Piritrexim | Burroughs | PCP treatment |
| | Wellcome | |
| Pentamidine | Fisons | PCP prophylaxis |
| isethionate | Corporation | |
| Spiramycin | Rhone-Poulenc | Cryptosporidial diarrhea |
| Intraconazole- | Janssen Pharm | Histoplasmosis; cryptococcal |
| R51211 | | meningitis |
| Trimetrexate | Warner-Lambert | PCP |

OTHER AGENTS

| Drug Name | Manufacturer | Therapeutic Use |
|---|---|---|
| Daunorubicin | NeXstar, Sequus | Karposi's sarcoma |
| Recombinant Human | Ortho Pharm. Corp. | Severe anemia assocated |
| Erythropoietin | | w/AZT therapy |
| Recombinant Human | Serono | AIDS-related wasting, |
| Growth Hormone | | cachexia |
| Megestrol Acetate | Bristol-Myers Squibb | Treatment of anorexia |
| | | associated w/AIDS |
| Testosterone | Alza, Smith Kline | AIDS-related wasting |
| Total Enteral | Norwich Eaton | Diarrhea and |
| Nutrition | Pharmaceuticals | malabsorption related to |
| | | AIDS |

The combinations of the anti-HCV compounds of this invention with AIDS antivirals, other antivirals, immunomodulators, anti-infectives, antibiotics, vaccines, other therapeutic agents are not limited to the list in the above Table, but includes, in principle, any combination with any pharmaceutical composition useful for the treatment against infection by HCV or for treating hepatitis C or for treating infections resulting from other viruses of Flaviviridae. Preferred combinations are simultaneous or alternating treatments of an anti-HCV compound of the present invention and a protease inhibitor (e.g., indinavir, nelfinavir, ritonavir, saqiiinavir and others), a reverse transcriptase inhibitor [nucleoside (e.g., AZT, 3TC, ddC, ddI, d4T, abacavir and others, and/or non-nucleoside (e.g., efavirenz, nevirapine, and others), or some combination of two or more of these inhibitors (see Table above). A few representative examples of relevant patents citing combinations are: EPO 0,484,071, U.S. Pat. No. 5,413,999, WO 9962513.

In such combinations, the compound of the present invention and other active agents may be separately administered or concurrently administered. In addition, the administration of one element may be prior to, concurrent to, or subsequent to the administration of other agent(s).

The following representative examples are provided to illustrate details for the preparation of the compounds of the present invention. The examples are not intended to be limitations on the scope of the present invention and they should not be so construed. Furthermore, the compounds described in the following examples are not to be viewed as forming the only set of compounds that is considered as the invention, and any combination of components of the compounds or their moieties may itself form a set. This has been addressed previously in this patent document. Those skilled in the art will readily comprehend that known variations of reaction conditions and synthetic conversions described in the following preparative procedures can be used to prepare these other compounds.

Chemical Synthesis

Chemical schemes for representative examples 1 through 12 are Schemes 1 and 2 shown below.

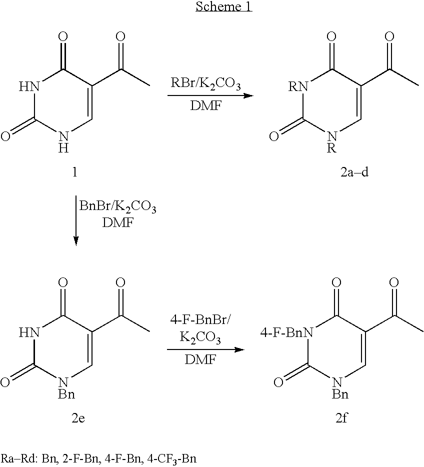

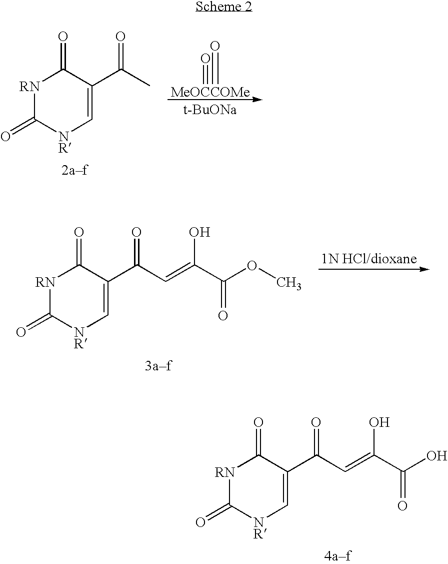

### REPRESENTATIVE EXAMPLE 1

### Methyl-4-(1,3-dibenzyl-1,2,3,4-tetrahydro-2,4-dioxopyrimidin-5-yl)-2-hydroxy4-oxobut-2-enoate (3a)

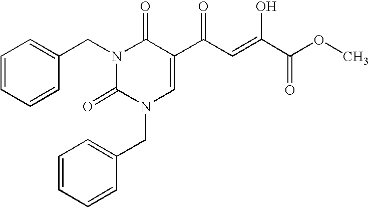

### Step 1: preparation of 5-acetyl-1,3-dibenzyluracil (2a)

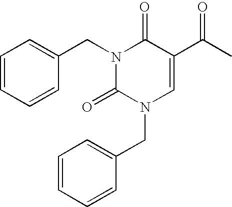

A suspension of 5-acetyluracil (3.1 g, 20 mmol), and potassium carbonate (6.9 g, 50 mmol) in DMF (75 ml) was stirred for 20 min. Then benzyl bromide (6.0 ml, 50 mmol) was added. The resulting mixture was stirred for 8 h at room temperature. DMF was distilled under vacuum. The residue was purified by column (dichloromethane:methanol 40:1). The appropriate fraction was concentrated and crystallized from ethanol to afford 5.34 g of a white solid. Yield was 79.8%. Mp. 92-93° C. ¹HNMR (CDCl₃): 8.23 (s, 1H), 7.29-7.49 (m, 10H), 5.17 (s, 2H), 5.01 (s, 2H), 2.62 (s, 3H). ¹³CNMR (CDCl₃): 194.5, 160.7, 151.0, 148.4, 136.2, 134.4, 129.2, 129.0, 128.9, 128.5, 128.2, 127.8, 112.2, 53.4, 44.9, 30.7. FAB-HRMS: [M+H]⁺ calcd. for C₂₀H₁₉N₂O₃ 335.1396, found 335.1412.

### Step 2: preparation of methyl 4-(1,3-dibenzyl-1,2,3,4-tetrahydro-2,4-dioxopyrimidin-5-yl)-2-hydroxy-4-oxobut-2-enoate (3a)

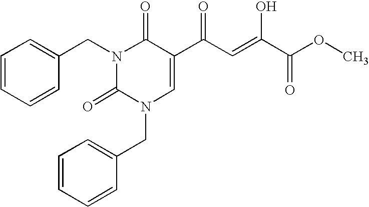

To a stirred solution of sodium t-butoxide (577 mg, 6 mmol) in anhydrous THF (15 ml) at room temperature was added, dropwise, dimethyl oxalate (472 mg, 4 mmol) in THF (7 ml) followed by 5-acetyl-1,3-dibenzyluracil (2a) (669 mg, 2 mmol) in THF (8 ml). The resulting mixture was stirred at room temperature for 4 h and then was acidified to pH=2. THF was evaporated. The residue in CH₂Cl₂ (100 ml) was washed with brine (20 ml) and purified by column chromatography (hexane:ethyl acetate, 2:1). The appropriate fraction was concentrated and crystallized from ethanol to give 254 mg of a yellow solid. Yield was 29.1%. Mp. 158-159° C. ¹HNMR (CDCl₃): 15.04 (s, br, 1H), 8.36 (s, 1H), 7.72 (s, 1H), 7.29-7.49 (m, 10H), 5.18 (s, 2H), 5.05 (s, 2H), 3.92 (s, 3H). ¹³CNMR (CDCl₃): 185.7, 168.8, 162.4, 159.7, 150.5, 148.5, 136.0, 134.0, 129.4, 129.1, 129.0, 128.5, 128.3, 127.9, 109.0, 101.6, 53.7, 53.2, 45.0. FAB-HRMS: [M+H]⁺ calcd. for C₂₃H₂₁N₂O₆ 421.1400, found 421.1418.

### REPRESENTATIVE EXAMPLE 2

### 4-(1,3-Dibenzyl-1,2,3,4-tetrahydro-2,4-dioxopyrimidin-5-yl)-2-hydroxy-4-oxobut-2-enoic acid (4a)

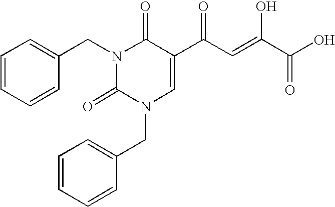

A solution of methyl 4-(1,3-dibenzyl-1,2,3,4-tetrahydro-2,4-dioxopyrimidin-5-yl)-2-hydroxy-4-oxobut-2-enoate (3a) (757 mg, 1.8 mmol) in dioxane (100 ml) was refluxed with 1N HCl (60 ml) for 4h. The solution was evaporated to dryness. The resulting solid was recrystalized from hexane and ethyl acetate (3:1) to give 617 mg a pale yellow solid. Yield was 84.2%. Mp. 186-188° C. ¹HNMR (DMSO-d6): 8.89 (s, 1H), 7.57 (s, 1H), 7.24-7.36 (m, 10H), 5.16 (s, 2H), 5.02 (s, 2H). “¹³CNMR (DMSO-d₆): 186.1, 169.0, 163.2, 159.9, 151.1, 150.2, 136.5, 135.8, 128.7, 128.4, 128.0, 127.8, 127.6, 127.3, 107.7, 100.9, 52.8, 44.2. FAB-HRMS: [M+H]⁺ calcd. for C₂₂H₁₉N₂O₆ 407.1243, found 407.1248.

### REPRESENTATIVE EXAMPLE 3

### Methyl 4-[1,3-bis(2-fluorobenzyl)-1,2,3,4-tetrahydro-2,4-dioxopyrimidin-5-yl]-2-hydroxy-4-oxobut-2-enoate (3b)

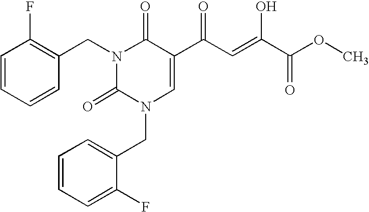

### Step 1: preparation of 1,3-bis(2-fluorobenzyl)-5-acetyluracil (2b)

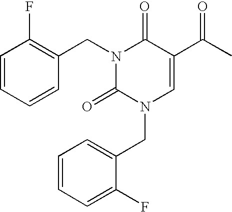

The title compound for this step was synthesized using a similar procedure to that described in Example 1, step 1, except that benzyl bromide was replaced with 2-fluorobenzyl bromide. The yield was 43.9%. Mp. 149-150° C. ¹HNMR (CDCl₃): 8.35 (d, 1H, J=1.0 Hz), 7.36-7.44 (m, 2H), 7.04-7.26 (m, 6H), 5.24 (s, 2H), 5.07 (s, 2H), 2.62 (s, 3H). ¹³CNMR (CDCl₃): 194.3, 161.1 (d, J=247.9 Hz), 160.7 (d, J=247.9 Hz), 160.6, 150.8, 148.8 (d, J=2.9 Hz), 131.3 (d, J=3.4 Hz), 130.9 (d, J=8.2 Hz), 129.19 (d, J=8.2 Hz), 129.17 (d, J=2.9 Hz), 124.7 (d, J=3.8 Hz), 124.1 (d, J=3.8 Hz), 123.1 (d, J=14.5 Hz), 121.4 (d, J=14.5 Hz), 115.9 (d, J=21.6 Hz), 115.5 (d, J=21.6 Hz), 112.2, 47.8, 38.8, 30.6.

FAB-HRMS: [M+H]⁺ calcd. for C₂₀H₁₇F₂N₂O₃ 371.1207, found 371.1202.

### Step 2: preparation of methyl 4-[1,3-bis(2-fluorobenzyl)-1,2,3,4-tetrahydro-2,4-dioxopyrimidin-5-yl]-2-hydroxy-4-oxobut-2-enoate (3b)

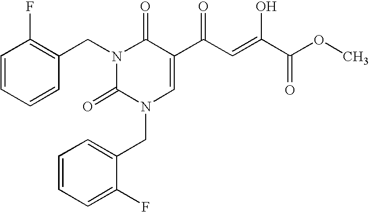

The title compound for this step was synthesized using a similar procedure to that described in the Example 1, step 2, except that 5-acetyl-1,3-dibenzyluracil was replaced with 1,3-bis(2-fluorobenzyl)-5-acetyluracil. The title compound was crystallized from the mixture of hexane and ethyl acetate (3:1) and obtained in 21.1% yield. Mp. 158-160° C. ¹HNMR(CDCl₃): 15.06 (br, s, 1H), 8.52 (s, 1H), 7.69 (s, 1H), 7.38-7.46 (m, 2H), 7.04-7.26 (m, 6H), 5.25 (s, 2H), 5.11 (s, 2H), 3.90 (s, 3H). ¹³CNMR(CDCl₃): 185.3, 169.2, 162.4, 161.2 (d, J=247.3 Hz), 160.7 (d, J=247.9 Hz), 159.6, 150.3, 148.9 (d, J=3.4 Hz), 131.5 (d, J=3.4 Hz), 131.2 (d, J=8.7 Hz), 129.3 (d, J=8.2 Hz), 129.2 (d, J=3.4 Hz), 124.8 (d, J=3.8 Hz), 124.1 (d, J=3.9 Hz), 122.8 (d, J=14.5 Hz), 121.2 (d, J=14.3 Hz), 116.0 (d, J=21.1 Hz), 115.6 (d, J=21.6 Hz), 108.9, 101.5, 53.0, 48.2 (d, J=3.4 Hz), 38.9 (d, J=4.8 Hz). FAB-HRMS: [M+H]⁺ calcd. for C₂₃H₁₉F₂N₂O₆ 457.1211, found 457.1203.

### REPRESENTATIVE EXAMPLE 4

### 4-[1,3-Bis(2-fluorobenzyl)-1,2,3,4-tetrahydro-2,4-dioxopyrimidin-5-yl]-2-hydroxy-4-oxobut-2-enoic acid (4b)

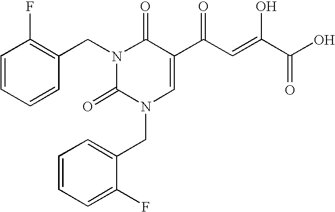

The title compound was synthesized using a similar procedure to that described in Example 2, except that methyl 4-(1,3-dibenzyl-1,2,3,4-tetrahydro-2,4-dioxopyrimidin-5-yl)-2-hydroxy-4-oxobut-2-enoate was replaced with methyl 4-[1,3-bis(2-fluorobenzyl)-1,2,3,4-tetrahydro-2,4-dioxopyrimidin-5-yl]-2-hydroxy-4-oxobut-2-enoate (3b). The title compound was crystallized from hexane and ethyl acetate (2:1) to give an off-white solid. The yield was 56.5%. Mp. 178-179 ° C. ¹H NMR(DMSO-d6): 15.00 (br, s, 1H), 14.02 (br, s, 1H), 8.90 (s, 1H), 7.55 (s, 1H), 7.08-7.40 (m, 8H), 5.23 (s, 2H), 5.05 (s, 2H). ¹³CNMR(DMSO-d6): 185.7, 169.2, 163.0, 160.2 (d, J=246.0 Hz), 159.8 (d, J=244.6 Hz), 159.7, 151.2, 149.9, 130.2, 129.0 (d, J=8.2 Hz), 128.4 (d, J=3.9 Hz), 124.5 (d, J=3.3 Hz), 124.3 (d, J=3.3 Hz), 123.1 (d, J=13.9 Hz), 122.3 (d, J=14.5 Hz), 115.4 (d, J=21.1 Hz), 115.1 (d, J=21.1 Hz), 107.6, 100.7, 47.8 (d, J=3.4 Hz), 38.2 (d, J=4.8 Hz).

FAB-HRMS: [M+H]⁺ calcd. for C₂₂H₁₇F₂N₂O₆443.1055, found 443.1045.

### REPRESENTATIVE EXAMPLE 5

### Methyl 4-[1,3-bis(4-fluorobenzyl)-1,2,3,4-tetrahydro-2,4-dioxopyrimidin-5-yl]-2-hydroxy-4-oxobut-2-enoate (3c)

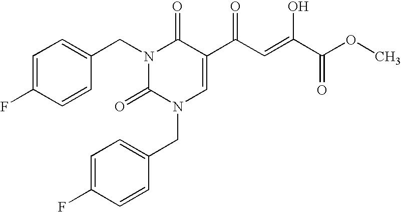

### Step 1: 1,3-bis(4-fluorobenzyl)-5-acetyluracil (2c)

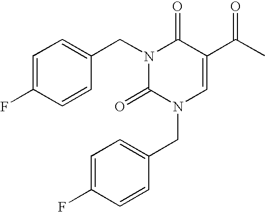

The title compound was synthesized using a similar procedure to that described in Example 1, step 1, except that benzyl bromide was replaced with 4-fluorobenzyl bromide. The yield was 51.8%. Mp. 134-135° C. ¹HNMR (CDCl₃): 8.22 (s, 1H), 7.48 (dd, 2H, J=9.0, 5.5 Hz), 7.32 (dd, 2H, J=8.5, 5.0 Hz), 6.99-7.09 (m, 4H), 5.11 (s, 2H), 4.97 (s, 2H), 2.62 (s, 3H). ¹³CNMR (CDCl₃): 194.3, 163.0 (d, J=248.3 Hz), 162.4 (d, J=246.4 Hz), 160.6, 150.9, 148.2, 132.1 (d, J=3.4 Hz), 131.1 (d, J=8.2 Hz), 130.23 (d, J=8.5 Hz), 130.26 (d, J=2.9 Hz), 116.2 (d, J=21.4 Hz), 115.3 (d, J=21.5 Hz), 112.4, 52.9, 44.2, 30.6. FAB-HRMS: [M+H]⁺ calcd. for C₂₀H₁₇F₂N₂O₃ 371.1207, found 371.1220.

### Step 2: preparation of methyl 4-[1,3-bis(4-fluorobenzyl)-1,2,3,4-tetrahydro-2,4-dioxopyrimidin-5-yl]-2-hydroxy-4-oxobut-2-enoate (3c)

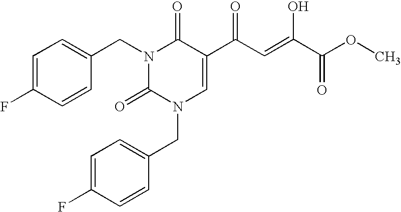

The title compound was synthesized using a similar procedure to that described in Example 1, step 2, except that 5-acetyl-1,3-dibenzyluracil was replaced with 1,3-bis(4-fluorobenzyl)-5-acetyluracil. The title compound was recrystallized from ethanol and obtained in 23.5% yield; Mp. 171-173° C. ¹HNMR (CDCl₃): 15.02 (br, s, 1H), 8.35 (s, 1H), 7.71 (s, 1H), 7.49 (m, 2H), 7.34 (m, 2H), 7.09 (m, 2H), 7.00 (m, 2H), 5.13 (s, 2H), 5.02 (s, 2H), 3.92 (s, 3H). ¹³CNMR (CDCl₃): 185.3, 169.2, 163.1 (d, J=248.8 Hz), 162.5 (d, J=246.4 Hz), 162.4, 159.6, 150.5, 148.2, 131.8 (d, J=3.4 Hz), 131.2 (d, J=8.2 Hz), 130.3 (d, J=8.7 Hz), 129.9 (d, J=3.4 Hz), 116.4 (d, J=21.6 Hz), 115.4 (d, J=21.6 Hz), 109.2, 101.5, 53.14, 53.12, 44.3. FAB-HRMS: [M+H]⁺ calcd. for C₂₃H₁₉F₂N₂O₆ 457.1211, found 457.1196.

### REPRESENTATIVE EXAMPLE 6

### 4-[1,3-Bis(4-fluorobenzyl)-1,2,3,4-tetrahydro-2,4-dioxopyrimidin-5-yl]-2-hydroxy-4-oxobut-2-enoic acid (4c)

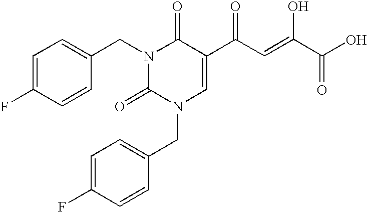

The title compound was synthesized using a similar procedure to that described in Example 2, except that methyl 4-(1,3-dibenzyl-1,2,3,4-tetrahydro-2,4-dioxopyrimidin-5-yl)-2-hydroxy-4-oxobut-2-enoate was replaced with methyl 4-[1,3-bis(4-fluorobenzyl)-1,2,3,4-tetrahydro-2,4-dioxopyrimidin-5-yl]-2-hydroxy-4-oxobut-2-enoate (3c). The title compound was crystalized from hexane and ethyl acetate (3:1). The yield was 49.7%. Mp. 186-188° C. ¹HNMR (DMSO-d₆): 15.07 (br, s, 1H), 14.02 (br, s, 1H), 8.90 (s, 1H), 7.56 (s, 1H), 7.34-7.46 (m, 4H), 7.10-7.21 (m, 4H), 5.13 (s, 2H), 4.98 (s, 2H). ¹³CNMR (DMSO-d₆): 185.8, 169.2, 163.1, 161.8 (d, J=244.1 Hz), 161.3 (d, J=243.2 Hz), 159.7, 150.8, 150.1, 132.6 (d, J=2.9 Hz), 131.8 (d, J=3.4.Hz), 130.2 (d, J=8.2 Hz), 129.9 (d, J=8.2 Hz), 115.4 (d, J=21.6 Hz), 115.0 (d, J=21.0 Hz), 107.7, 100.7, 52.1, 43.5. FAB-HRMS: [M+H]⁺ calcd. for C₂₂H₁₇F₂N₂O₆ 443.1055, found 443.1044.

### REPRESENTATIVE EXAMPLE 7

### Methyl 4-[1,3-bis(4-(trifluoromethyl)benzyl)-1,2,3,4-tetrahydro-2,4-dioxopyrimidin-5-yl]-2-hydroxy-4-oxobut-2-enoate (3d).

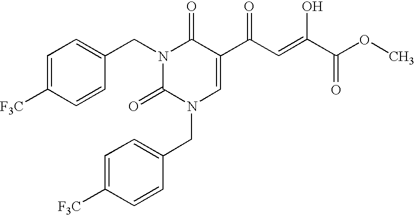 cl Step 1: preparation of 1,3-bis(4-(trifluoromethyl)benzyl)-5-acetyluracil (2d)

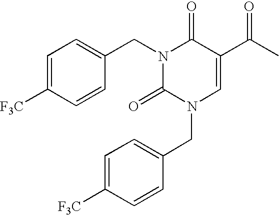

The title compound was synthesized using a similar to that described in the Example 1, step 1, except that benzyl bromide was replaced with 4-(trifluoromethyl)benzyl bromide. The yield was 65.1%, amorphous solid. ¹HNMR (CDCl₃): 8.27 (s, 1H), 7.44-7.66 (m, 8H), 5.20 (s, 2H), 5.07 (s, 2H), 2.63 (s, 3H). ¹³CNMR (CDCl₃): 194.1, 160.5, 150.9, 148.3, 140.0, 138.2, 131.3 (q, J=32.7 Hz), 130.2 (q, J=32.7 Hz), 129.3, 128.4, 126.3 (q, J=3.8 Hz), 125.5 (q, J=3.8 Hz), 124.0 (q, J=272.0 Hz), 123.7 (q, J=272.3 Hz), 112.6, 53.2, 44.5, 30.6. FAB-HRMS: [M+H]⁺ calcd. for C₂₂H₁₇F₆N₂O₃ 471.1143, found 471.1148.

### Step 2: preparation of methyl 4-[1,3-bis(4-(trifluoromethyl)benzyl)-1,2,3,4-tetrahydro-2,4-dioxopyrimidin-5-yl]-2-hydroxy-4-oxobut-2-enoate (3d)

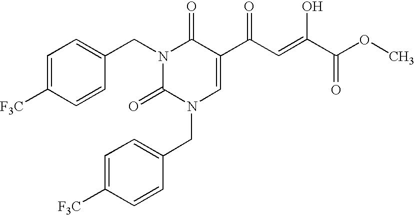

The title compound was synthesized using a similar procedure to that described in Example 1, step 2, except that 5-acetyl-1,3-dibenzyluracil was replaced with 1,3-bis(4-(trifluoromethyl)benzyl)-5-acetyluracil. The title compound crystallized from a mixture of hexane and ethyl acetate (3:1) and ethanol and was obtained in 20.3% yield. Mp. 189-191° C. ¹H NMR (CDCl₃): 14.98 (br, s, 1H), 8.41 (s, 1H), 7.70 (s, 1H), 7.46-7.68 (m, 8H), 5.21 (s, 2H), 5.11 (s,2H). ¹³CNMR(CDCl₃): 185.0, 169.5, 162.3, 159.5, 150.4, 148.3, 139.7, 138.0, 131.4 (q, J=32.5 Hz), 130.3 (q, J=32.4 Hz), 129.4, 128.5, 126.3 (q, J=3.7 Hz), 125.5 (q, J=3.7 Hz), 124.0 (q, J=271.9 Hz), 123.7 (q, J=272.3 Hz), 109.4, 101.5, 53.4, 53.2, 44.6. FAB-HRMS: [M+H]⁺ calcd. for C₂₅H₁₉F₆N₂O₆ 557.1147, found 557.1135.

### REPRESENTATIVE EXAMPLE 8

### 4-[1,3-Bis(4-(trifluoromethyl)benzyl)-1,2,3,4-tetrahydro-2,4-dioxopyrimidin-5-yl1-2-hydroxy-4-oxobut-2-enoic acid (4d)

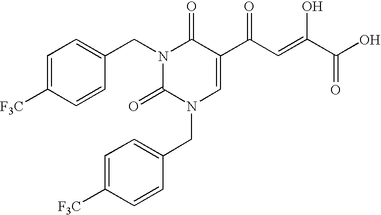

The title compound was synthesized using a similar procedure to that described in Example 2, except that methyl 4-(1,3-dibenzyl-1,2,3,4-tetrahydro-2,4-dioxopyrimidin-5-yl)-2-hydroxy-4-oxobut-2-enoate was replaced with methyl 4-[1,3-bis(4-(trifluoromethyl)benzyl)-1,2,3,4-tetrahydro-2,4-dioxopyrimidin-5-yl]-2-hydroxy-4-oxobut-2-enoate (3d). The title compound was recrystalized from hexane and ethyl acetate (3:1). The yield was 68.2%. Mp. 176-178° C. ¹HNMR (DMSO-d₆): 14.98 (br, s, 1H), 14.02 (br, s, 1H), 8.99 (s, 1H), 7.72 (d, 2H, J=8.0 Hz), 7.66 (d, 2H, J=8.5 Hz), 7.59 (d, 2H, J=8.5 Hz), 7.57 (s, 1H), 7.51 (d, 2H, J=8.0 Hz), 5.26 (s, 2H), 5.09 (s, 2H). ¹³CNMR (DMSO-d₆): 185.8, 169.3, 163.1, 159.9, 151.3, 150.2, 141.2, 140.5, 128.4 (q, J=31.5 Hz), 128.3, 128.2, 127.9 (q, J=31.7 Hz), 125.4 (q, J=3.8 Hz), 125.2 (q, J=3.8 Hz), 124.2 (q, J=272.3 Hz), 124.1 (q, J=271.8 Hz), 108.0, 100.7, 52.6, 43.9. FAB-HRMS: [M+H]⁺ calcd. for C₂₄H₁₇F₆N₂O₆ 543.0991, found 543.1003.

### REPRESENTATIVE EXAMPLE 9

### Methyl 4(1-benzyl-1,2,3,4-tetrahydro-2,4-dioxopyrimidin-5-yl)-2-hydroxy-4-oxobut-2-enoate (3e)

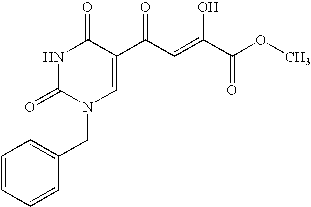

### Step 1: preparation of 5-acetyl-1-benzyluracil (2e)

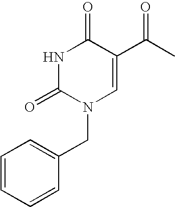

The title compound was synthesized in 69.9% yield by a similar procedure to that described for Example 1, step 1, but using 1.1 equiv of benzyl bromide and 1.0 equiv of potassium carbonate in DMF. Mp. 196-197° C. ¹HNMR (DMSO-d₆): 11.69 (br, s, 1H), 8.54 (s, 1H), 7.30-7.36 (m, 5H), 5.03 (s, 2H), 2.44 (s, 3H). ¹³CNMR (DMSO-d6): 193.5, 161.6, 151.5, 150.3, 136.2, 128.7, 127.9, 127.7, 111.8, 51.1, 30.3: FAB-HRMS: [M+H]⁺ calcd. for C₁₃H₁₃N₂O₃ 245.0926, found 245.0932.

### Step 2: preparation of methyl 4-(1-benzyl-1,2,3,4-tetrahydro-2,4-dioxopyrimidin-5-yl)-2-hydroxy-4-oxobut-2-enoate (3e)

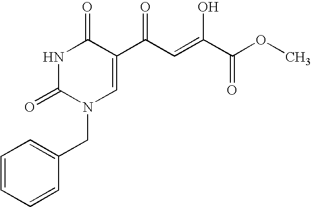

The title compound was synthesized using a similar procedure to that described in Example 1, step 2, except that 5-acetyl-1,3-dibenzyluracil was replaced with 5-acetyl-1-benzyluracil. The title compound was crystallized from ethanol and obtained in 77.2% yield. Mp. 197-199° C. ¹HNMR (DMSO-d₆): 11.90 (s, 1H), 8.82 (s, 1H), 7.57 (s, 1H), 7.31-7.37 (m, 5H), 5.08 (s, 2H), 3.82 (s, 3H). ¹³CNMR (DMSO-d₆): 185.9, 167.8, 162.2, 161.0, 152.7, 149.8, 135.9, 128.7, 127.9,. 127.7, 107.9, 100.9, 53.0, 51.5. FAB-HRMS: [M+H]⁺ calcd. for C₁₆H₁₅N₂O₆ 331.0930, found 331.0928.

### REPRESENTATIVE EXAMPLE 10

### 4-(1-Benzyl-1,2,3,4-tetrahydro-2,4-dioxopyrimidin-5-yl)-2-hydroxy-4-oxobut-2-enoic acid (4e)

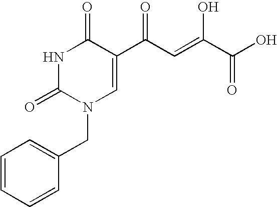

The title compound was synthesized using a similar procedure to that described in Example 2, replacing methyl 4-(1,3-dibenzyl-1,2,3,4-tetrahydro-2,4-dioxopyrimidin-5-yl)-2-hydroxy-4-oxobut-2-enoate with methyl 4-(1-benzyl-1,2,3,4-tetrahydro-2,4-dioxopyrimidin-5-yl)-2-hydroxy-4-oxobut-2-enoate (3e). The title compound was crystallized from mixture of tetrahydofuran and chloroform (2:3). The yield was 79.7%. Mp. 195-197° C. ¹HNMR (DMSO-d₆): 15.10 (br, s, 1H), 13.97 (br, s, 1H), 11.87 (s, 1H), 8.79 (s, 1H), 7.54 (s, 1H), 7.30-7.36 (m, 5H), 5.08 (s, 2H). ¹³CNMR (DMSO-d₆): 186.0, 169.2, 163.2, 161.0, 152.5, 149.9, 136.0, 128.7, 127.9, 127.7, 108.2, 100.8, 51.5. FAB-HRMS: [M+H]⁺ calcd. for C₁5H₁₃N₂O₆ 317.0774, found 317.0769.

### REPRESENTATIVE EXAMPLE 11

### Methyl 4-[3-(4-fluorobenzyl)-1-benzyl-1,2,3,4tetrahydro-2,4-dioxopyrimidin-5-yl]-2-hydroxy-4-oxobut-2-enoate (3f)

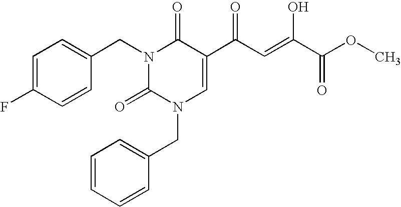

### Step 1: preparation of 3-(4-fluorobenzyl)-5-acetyl-1-benzyluracil (2f)

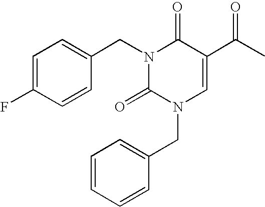

The title compound was synthesized in 93.7% yield by benzylation of 5-acetyl-1-benzyluracil (2e) with 2 equiv of 4-fluorobenzyl bromide and 2 equiv of potassium carbonate in DMF. Mp. 106-108° C. ¹HNMR (CDCl₃): 8.23 (s, 1H), 7.30-7.50 (m, 7H), 7.00 (m, 2H), 5.12 (s, 2H), 5.01 (s, 2H), 2.61 (s, 3H). ¹³CNMR (CDCl₃): 194.3, 162.4 (d, J=246.4 Hz), 160.7, 151.0, 148.4, 134.4, 132.1 (d, J=3.4 Hz), 131.1 (d, J=8.2 Hz), 129.2, 128.9, 128.2, 115.3 (d, J=21.6 Hz), 112.3, 53.4, 44.2, 30.6. FAB-HRMS: [M+H]⁺ calcd. for C₂₀RH₈FN₂O₃ 353.1301, found 353.1310.

### Step 2: preparation of methyl 4-[3-(4-fluorobenzyl)-1-benzyl-1,2,3,4-tetrahydro-2,4-dioxopyrimidin-5-yl]-2-hydroxy-4-oxobut-2-enoate (3f)

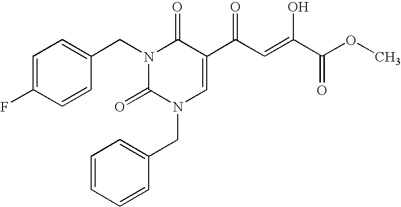

The title compound was synthesized using a similar procedure to that described Example 1, step 2, replacing 5-acetyl-1,3-dibenzyluracil with 3-(4-fluorobenzyl)-5-acetyl-1-benzyluracil. The title compound was crystallized from ethanol and obtained in 30.5% yield. Mp. 165-167° C. ¹HNMR (CDCl₃): 15.04 (br, s, 1H), 8.36 (s, 1H), 7.72 (s, 1H), 7.28-7.52 (m, 7H), 7.01 (t, 2H, J=8.5 Hz), 5.15 (s, 2H), 5.06 (s, 2H), 3.93 (s, 3H). ¹³CNMR (CDCl₃): 185.4, 169.2, 162.5 (d, J=246.8 Hz), 162.4, 159.7, 150.5, 148.4, 134.1, 131.9 (d, J=3.4 Hz), 131.2 (d, J=8.2 Hz), 129.4, 129.1, 128.3, 115.4 (d, J=21.6 Hz), 109.1, 101.5, 53.7, 53.1, 44.3. FAB-HRMS: [M+H]⁺ calcd. for C₂₃H₂₀FN₂O₆ 439.1305, found 439.1294.

### REPRESENTATIVE EXAMPLE 12

### 4-[3-(4-Fluorobenzyl)-1-benzyl-1,2,3,4-tetrahydro-2,4-dioxopyrimidin-5-yl]-2-hydroxy-4-oxobut-2-enoic acid (4f)

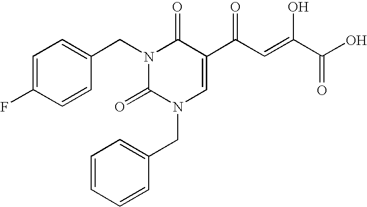

The title compound was synthesized using a similar procedure to that described in Example 2, replacing methyl 4-(1,3-dibenzyl-1,2,3,4-tetrahydro-2,4-dioxopyrimidin-5-yl)-2-hydroxy-4-oxobut-2-enoate with methyl 4-[3-(4-fluorobenzyl)-1-benzyl-1,2,3,4-tetrahydro-2,4-dioxopyrimidin-5-yl]-2-hydroxy-4-oxobut-2-enoate (3f). The title compound was crystallized from a mixture of hexane and ethyl acetate (2:1). The yield was 64.0%. Mp. 188-190° C. ¹HNMR (DMSO-d6): 15.05 (br, s, 1H), 14.01 (br, s, 1H), 8.87 (s, 1H), 7.56 (s, 1H), 7.30-7.37 (m, 7H), 7.12 (m, 2H), 5.15 (s, 2H), 4.99 (s, 2H). ¹³CNMR (DMSO-d₆): 185.7, 169.2, 163.1, 161.4 (d, J=243.0 Hz), 159.7, 150.8, 150.1, 135.6, 132.6 (d, J=3.4 Hz), 129.9 (d, J=8.2 Hz), 128.6, 127.9, 127.7, 115.5 (d, J=21.6 Hz), 107.7, 100.7, 52.7, 43.5. FAB-HRMS: [M+H]⁺ calcd. for C₂₂H₁₈FN₂O₆ 425.1149, found 425.1156.

### REPRESENTATIVE EXAMPLE 13

### 4-(9-Benzyl-9H-purin-6-yl)-2-hydroxy-4-oxo-but-3-enoic acid (1l)

The relevant scheme is Scheme 3 shown below.

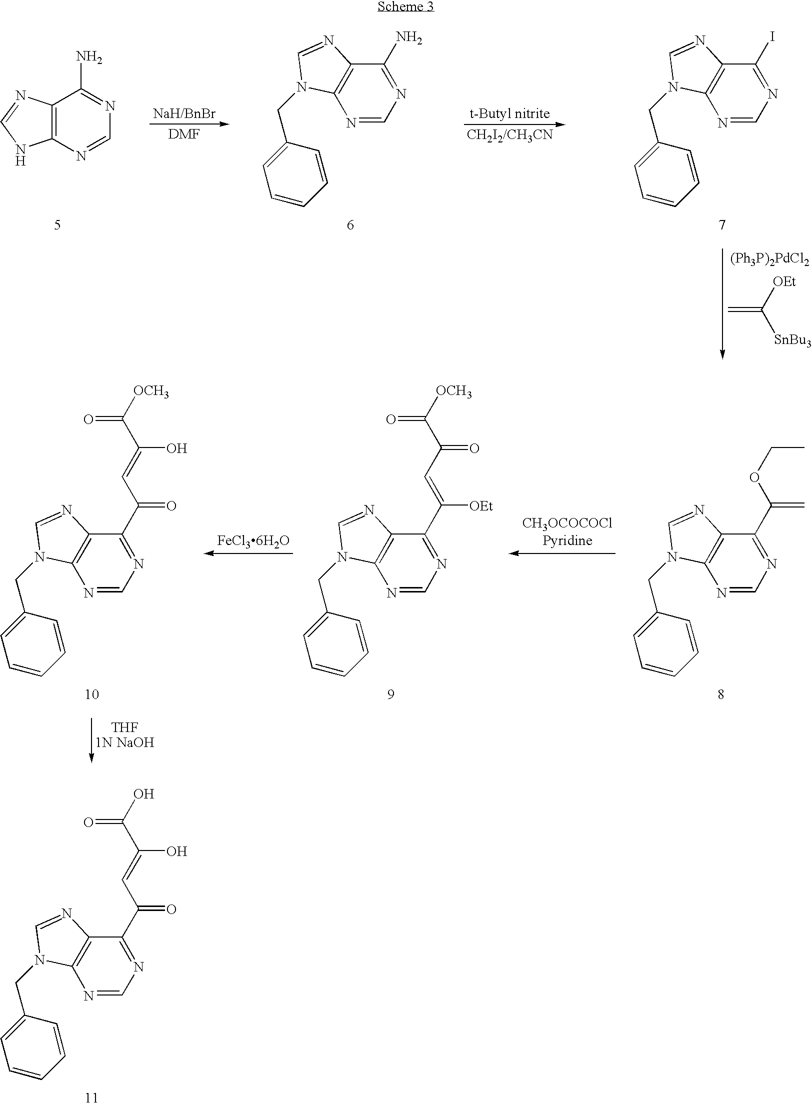

### Step 1: 9-Benzyladenine (6)

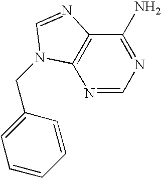

To a suspension of adenine (5) (5.00 g, 37.0 mmol) in dry DMF (120 mL) was added NaH (1.77 g, 44.4 mmol) at room temperature. The reaction mixture was stirred for 30 min and the resulting white suspension was warmed to 60° C. for an additional 30 min. Benzyl bromide (7.59 g, 44.4 mmol) was added and the mixture was stirred for 24 h at 60° C. TLC of the reaction mixture indicated the formation of two products. DMF was distilled off under reduced pressure and the resulting residue was treated with water (20 mL). The white solid that separated out, was filtered and dried under vaccum. Separation and purification was through flash column chromatography using CHCl₃:MeOH (9:1) for elution. 9-Benzyladenine:yield 5.5 g. (66%); mp 231-232° C.; ¹H NMR (DMSO-d₆,: δ 5.38 (s, 2H, CH₂), 7.29-7.33 (m, 7H, Ar—H and NH₂), 8.17 (s, 1H, purine C₈—H), 8.28 (s, 1H, purine C₂—H). 7-Benzyladenine: yield 1.8 g. (21%). Mp 252-255° C. ¹H NMR (DMSO-d₆): δ 5.53 (s, 2H, CH₂), 7.29-7.48 (m, 5H, Ar—H), 7.81 (s, 1H, purine C₈—H), 8.0-8.1 (br, 2H, NH₂), 8.6 (s, 1H, purine C₂—H).

### Step 2: 9-Benzyl-6-iodopurine (7)

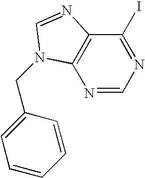

To a stirred suspension of 9-benzyladenine (6) (1.00 g, 4.4 mmol) in anhydrous acetonitrile (50 mL), was added diidomethane (5.82 g, 21.7 mmol) followed by t-butyl nitrite (2.24 g, 21.7 mmol) at 0-5° C. (ice bath). While maintaining the cooling, the solution was purged with nitrogen for 30 min. The ice bath was removed and the reaction mixture was heated under an atmosphere of nitrogen at 60-65° C. for 5 h. Acetonitrile and the excess reagents were distilled off and the residue obtained redissolved in chloroform (100 mL) and washed with saturated aqueous sodium sulfite (2×50 mL) followed by brine solution (2×50 mL). The chloroform layer was dried over anhydrous sodium sulfate and concentrated to give a reddish oil which was purified by flash chromatography on silica gel using EtOAc/hexane (3:7) for elution. Yield: 0.519 g. (34%). Mp 152-153° C. ¹H NMR (DMSO-d6): 8 5.51 (s, 2H, CH₂), 7.31-7.36 (m, 5H, Ar—H), 8.65 (s, 1H, purineC₈—H), 8.83 (s, 1H, purine C₂—H). ¹³C NMR (DMSO-d₆): δ 47.4, 123.2, 128.1, 128.1, 128.4, 129.2, 129.2, 136.5, 138.4, 146.9, 148.3, 152.4.

### Step 3: 9-Benzyl-6-(α-ethoxyvinyl)purine (8)

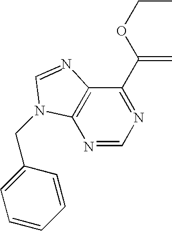

A mixture of 9-benzyl-6-iodopurine (7) (1.00 g, 2.8 mmol), bis(triphenylphosphine)-palladium(II) chloride (0.208 g, 0.02 mmol) and ethoxyvinyl(tributyl)tin (2.07 g, 3.8 mmol) in dry DMF (4 mL) was heated under N₂ at 100° C. for 6 h. TLC indicated completion of reaction. DMF was distilled off under reduced pressure and the resulting residue was redissolved in EtOAc (50 mL) and filtered through a pad of celite. The solvent (EtOAc) was distilled off and the residue obtained purified by flash chromatography. Yield 0.393 g, (47%). Mp 114-115° C. ¹H NMR (CDCl₃): δ 1.55 (t, 3H, CH₃, J =7.5 Hz), 4.13 (q, 2H, CH₂, J=13.7 Hz), 4.99 (d, 1H, CH, J=3Hz), 5.48 (s, 2H, benzylic CH₂), 6.16 (d, 1H, CH, J=3 Hz), 7.30-7.38 (m, 5H, Ar—H), 8.09 (s, 1H, purine C₈—H), 9.07 (s, 1H, purine C₂—H). ¹³C NMR (CDCl₃): 814.3, 47.3, 63.7, 94.7, 127.8, 127.8, 128.6, 129.1, 129.2, 130.3, 135.0, 144.4, 152.1, 152.3, 152.4, 155.4.

### Step 4: Methyl 4-(9-benzyl-9H-purin-6-yl)-4-ethoxy-2-oxo-but-3-enoate (9)

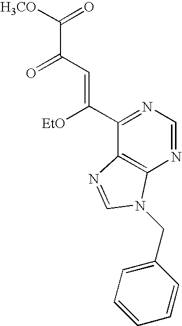

To a stirred solution of 9-benzyl-6-(α-ethoxyvinyl)purine (8) (0.20 g, 0.70 mmol ) and pyridine (0.688 mL, 0.72 g, 28.5 mmol ) in dry chloroform (10 mL ) at 0° C. was added methyl chlorooxoacetate (1.048 g, 0.784 mL, 28.5 mmol ) in dry chloroform (5 mL). The reaction mixture was allowed to attain ambient temperature, stirred for 3 days and then washed with water (2×10 mL) and dried over anhydrous sodium sulfate. The solvent was distilled off and the dark reddish syrup was purified by column chromatograpy. Yield 110 mg, (42% ). ¹H NMR (CDCl₃): 1.53 (t, 3H, CH₃, J=6.5 Hz), 3.80 (s, 3H, CH₃), 4.36 (q, 2H, CH₂, J=6.5 Hz), 5.49 (s, 2H, benzylic CH₂), 6.72 (s, 1H, olefmic CH), 7.36 (m, 5H, Ar—H), 8.07 (s, 1H, purine C₈—H), 9.10 (s, 1H, purine C₂—H). 13C NMR (CDCl₃): δ 14.1, 31.0, 47.5, 52.9, 67.0, 99.6, 128.0, 128.0, 128.8, 129.3, 129.3, 131.3, 134.7, 145.5, 152.0, 152.6, 162.4, 167.4, and 179.7.

### Step 5: Methyl 4-(9-benzyl-9H-purin-6-yl)-2-hydroxy-4-oxo-but-2-enoate (10)

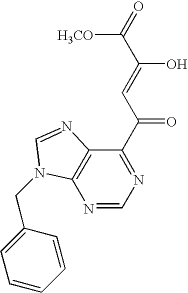

Methyl 4-(9-benzyl-9H-purin-6-yl)-4-ethoxy-2-oxo-but-3-enoate (9) (100 mg, 0.20 mmole ) obtained in above step was stirred at room in CH₂Cl₂ (20 mL) and treated with FeCl₃.6H₂O (0.125 g, 0.40 mmole). The reaction mixture was stirred at 40° C. for 5 h. Chloroform was distilled off and the resulting residue was treated with 1 N HCl (50 mL) for 1 h and then extracted with EtOAc (4×20 mL). The extract was dried over anhydrous sodium sulfate and the EtOAc distilled off to give a brownish residue which was purified by ion exchange chromatography (diethylamino sephadex anion exchange resin, CH₃CN:H₂O, (1:1) eluent). Yield 5.2 mg. Mp 166-167° C. ¹H NMR (CDCl₃) δ 3.99 (s, 3H, CH₃), 5.54 (s, 2H, benzylic CH₂), 7.35-7.41 (m, 5H, aromatic), 7.9 (s, 1H, olefinic CH), 8.3 (s,1H, purine C₈—H), 9.19 (s,1H, purine C₂—H); ¹³C NMR (CDCl₃) δ 47.7, 53.4, 101.4, 128.0, 128.0, 128.9, 129.3, 131.8, 134.5, 147.4, 152.3, 154.3, 162.1, 172.8, and 185.7. FAB-HRMS: [M+H]⁺ calcd. for C₁₇H₁₅N₄O₄ 339.1093, found 339.1083.

### Step 6: Synthesis of4-(9-benzyl-9H-purin-6-yl)-2-hydroxy-4-oxo-but-3-enoic acid (11)

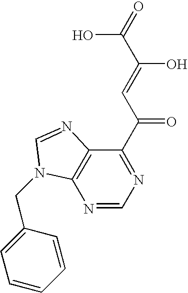

To a stirred solution of methyl 4-(9-benzyl-9H-purin-6-yl)-2-hydroxy-4-oxo-but-3-enoate (10) (17 mg, 0.05 mmol) in THF (5 mL) at 0° C. was added a solution of 1N NaOH (0.5 mL) and the reaction mixture was allowed to stir at 0° C. for 2 h. The reaction mixture was extracted with diethyl ether (2×10 mL) and the aqueous layer was acidified with dilute HCl and extracted with ethyl acetate (2×25 mL). The organic extract was washed with brine solution, dried over anhydrous sodium sulfate and concentrated. The crude solid was purified by trituration with diethyl ether to give 4 mg of product. Yield 25%. Mp 152-153° C. ¹H NMR (CDCl₃): δ 5.27 (s, 2H, benzylic CH₂), 6.39 (s, 1H, olefenic CH,), 7.23-7.29 (m, 5H, Ar—H), 8.78 (s, 1H, purine C₈—H), 8.84 (s, 1H, purine C₂—H). EIMS (m/z): [M+1] calc for C₁₆H₁₃N₄O₄ 325, found 325.

### REPRESENTATIVE EXAMPLE 14

### 4-(9-Benzyl-9H-purin-8-yl)-2-hydroxy4-oxobut-2-enoic acid (17)

The relevant scheme is Scheme 4 shown below.

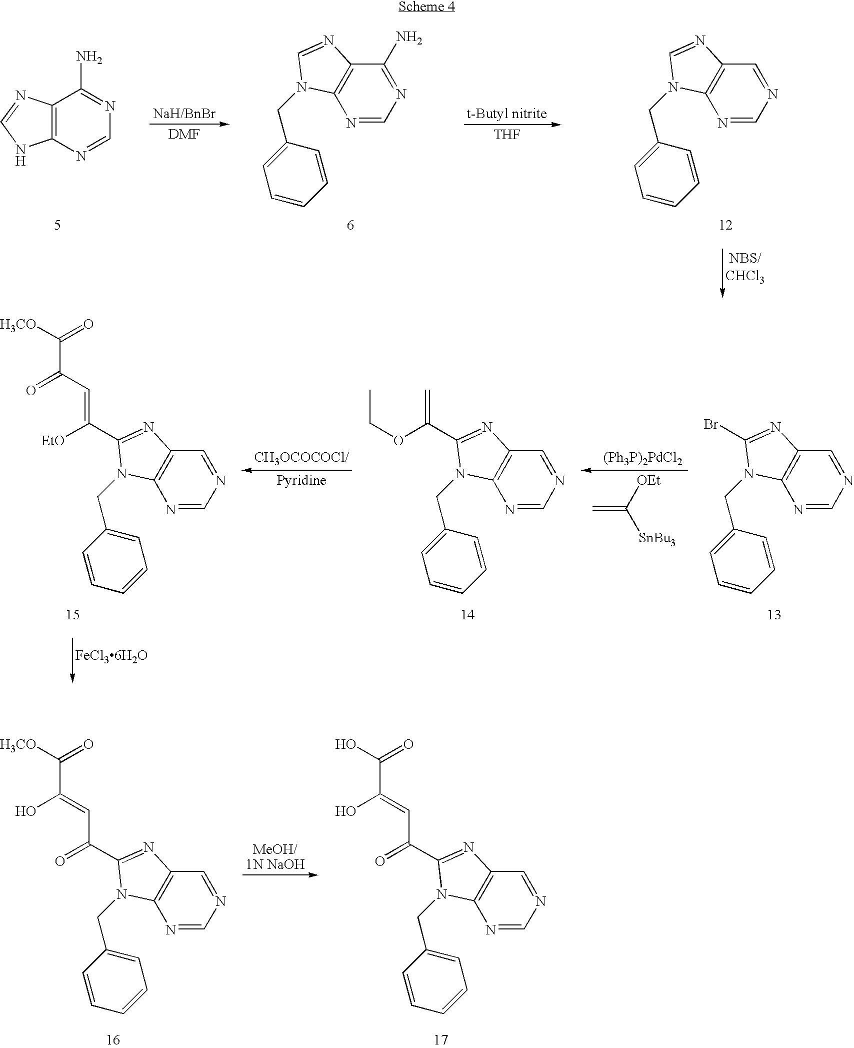

### 4-(9-Benzyl-9H-purin-8-yl)-2-hydroxy-4-oxobut-2-enoic acid. (17)

### Step 1. Described in step 1 of Example 13

### Step 2: Synthesis of 9-benzylpurine (12)

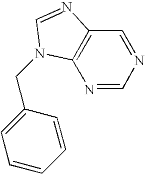

To a stirred suspension of 9-benzyladenine (6) (22.0 g, 97.6 mmol) in anhydrous THF (500 mL) was added t-butyl nitrite (9.34 g, 478.5 mmol) and the reaction mixture heated under an atmosphere of nitrogen at 60-65° C. for 4 h. THF and the excess reagent were distilled off and the residue obtained redissolved in chloroform (100 mL) and washed with brine solution (2×50 mL). The chloroform layer was dried over anhydrous sodium sulfate and distilled off to give a reddish oil, which was purified by flash chromatography on silica gel using EtOAc/hexane (8:2) for elution. Yield 10.68 g (42.3%). Mp 99-100° C. ¹H NMR (CDCl₃): δ 5.49 (s, 2H, CH₂), 7.34-7.40 (m, 5H, Ar—H), 8.10 (s, 1H, purine C₆—H), 9.06 (s, 1H, purine C₈H), 9.20 (s, 1H, purine C₂H).

### Step 3: 9-Benzyl-8-bromo-9H-purine (13)

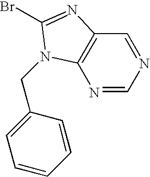

To a stirred solution of 12 (10.68 g, 50.7 mmol) in chloroform (500 mL) was added N-bromosuccinamide (45.20 g, 253.9 mmol) and the reaction mixture stirred under an atmosphere of nitrogen and at reflux temperature for 5 h. The reaction mixture was transferred to a separatory funnel and washed with saturated sodium sulfite solution (2×250 mL) followed by brine solution (2×250 mL). The chloroform fraction was dried over anhydrous sodium sulfate and concentrated and the reddish oil was purified by flash chromatography on silica gel using EtOAc/hexane (4:6) for elution. Yield 6.05 g. (41.2%). Mp 119-121° C.; ¹H NMR (CDCl₃): δ 5.53 (s, 2H, CH₂), 7.35-7.39 (m, 5H, Ar—H), 9.03 (s, 1H, purine C₈—H), 9.09 (s, 1H, purine C₂—H).

### Step 4: 9-Benzyl-8-(α-ethoxyvinyl)purine (14)

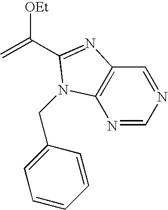

A mixture of 9-benzyl-8-bromopurine 13 (1.0 g, 3.4 mmol) bis(triphenylphosphine)-palladium(II)chloride (0.242 g, 0.30 mmol) and ethoxyvinyl(tributyl)tin (1.49 g, 4.14 mmol) in dry DMF (50 mL) was heated under N₂ at 65° C. for 48 h. DMF was distilled off under reduced pressure and the resulting residue was redissolved in EtOAc (50 mL) and filtered through a pad of celite. The EtOAc was distilled off and the residue obtained was purified by flash chromatography. Yield 0.579 g, (59.7%). ¹H NMR (CDCl₃): δ 1.33 (t, 3H, CH₃, J=7.5 Hz), 3.99 (q, 2H, CH₂, J=13.7 Hz), 4.66 (d, 1H, CH, J=3Hz), 5.34 (d, 1H, CH, J=3 Hz), 5.48 (s, 2H, benzylic CH₂), 7.30-7.38 (m, 5H, Ar—H), 9.09 (s, 1H, purine C₈—H), 9.2 (s, 1H, purine C₂—H). ¹³C NMR (CDCl₃): δ 14.2, 47.3, 64.2, 91.8, 126.8, 126.8, 127.7, 128.6, 128.6, 133.1, 136.4, 148.2, 151.2, 151.9,.152.8, 153.0.

### Step 5: Synthesis of methyl 4-(9-benzyl-9H-purin-8-yl)-4-ethoxy-2-oxo-but-3-enoate (15)

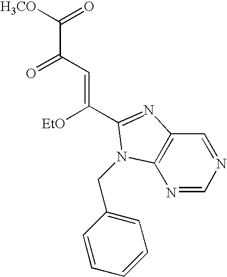

To a stirred solution of 9-benzyl-8-(α-ethoxyvinyl)purine (14) (0.579 g, 2.0 mmol ) and pyridine (2.08 g, 24.7 mmol ) in dry chloroform (15 mL ) at 0° C. was added methyl chlorooxoacetate (3.03 g, 24.7 mmol ) in dry chloroform (10 mL). The reaction mixture was allowed to stand in the refrigerator for 15 h. and then washed with (2×20 mL ) water and the organic layer dried over anhydrous sodium sulfate. Removal of chloroform gave a dark reddish syrup which was purified by column chromatography. Yield 0.538 g (77% ). ¹H NMR (CDCl₃): 1.17 (t, 3H, CH₃, J=6.5 Hz), 3.68 (s, 3H, CH₃), 3.93 (q, 2H, CH₂, J=6.5 Hz), 5.35 (s, 2H, benzylic CH₂), 6.45 (s, 1H, olefinic CH), 7.12-7.22 (m, 5H, Ar—H), 8.99 (s, 1H, purine C₆—H), 9.08 (s, 1H, purine C₂—H). 13C NMR (CDCl₃): o 13.7, 46.8, 53.2, 67.2, 102.2, 127.8, 128.3, 128.7, 130.9, 133.0, 135.2, 149.1, 149.4, 152.1, 153.5, 162.1, 180.3.

### Step 6: Methyl 4-(9-benzyl-9H-purine-8-yl)-2-hydroxy-4-oxo-but-3-enoate (16)

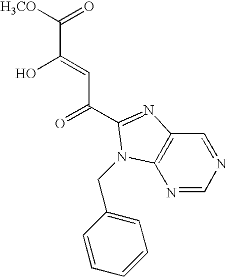

Methyl-4-(9-benzyl-9H-purin-6-yl)-4-ethoxy-2-oxo-but-3-enoate (15) (210 mg, 0.50 mmole) obtained in above step was stirred at room temperature in CH₂Cl₂ (60 mL) and treated with FeCl₃.6H₂O (0.262 g, 0.9 mmole). The reaction mixture stirred at 40° C. for 6 h and concentrated and the residue obtained was treated with 1 N HCl (50 mL) for 5 min and extracted with EtOAc (4×20 mL). The organic layer was dried over anhydrous sodium sulfate and concentrated to give a yellowish residue which was purified by ion exchange chromatography. Yield 90 mg. (46%). Mp 137-138° C.; ¹H NMR (CDCl₃) δ 3.98 (s, 3H, CH₃), 6.03 (s, 2H, benzylic CH₂), 7.29-7.41 (m, 5H, aromatic ), 7.68 (s, 1H, olefinic CH), 9.21 (s, 1H, purine C₆—H), 9.39 (s, 1H, purine C₂—H). 13C NMR (CDCl₃) δ 47.6, 53.5, 102.2, 128.0, 128.0, 128.1, 128.3, 128.8, 132.8, 135.9, 146.8, 151.6,152.5, 155.3, 161.9, and 186.2. FAB-HRMS: [M+H]⁺ calcd for C₁₇H₁₅N₄O₄ 339.1093, found 339.1099.

### Step 7: Synthesis of 4-(9-benzyl-9H-purine-8-yl)-2-hydroxy-4-oxo-but-3-enoic acid (17)

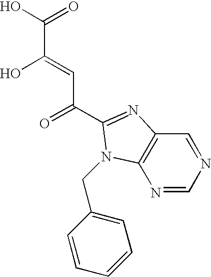

To a stirred solution of methyl 4-(9-benzyl-9H-purin-8-yl)-4-ethoxy-2-oxo-but-3-enoate (16) (0.020 g, 0.059 mmol) in MeOH (3 mL) at 0° C. was added a solution of 1N NaOH (1 mL) and reaction mixture allowed to stir at 0° C. for 30 min and then at ambient temperature for 30 min. The reaction mixture was neutralized with 1 N HCl and the precipitated solid was filtered dried and triturated with chloroform to give yellow solid. Yield: 14 mg (73%). Mp 162-163° C. ¹H NMR (DMSO-d₆): 5.90 (s, 2H, benzylic CH₂), 7.26-7.37 (m, 6H, Ar—H and olefinic H), 9.16 (s,1H, purine C₆—H), 9.49 (s, 1H, purine C₂—H). NMR (CDCl₃): δ 47.6, 101.5, 124.7, 126.6, 127.5, 127.5, 127.5, 128.8, 128.9, 128.9, 137.1, 137.5, 153.0, 155.2, 163.9, 192.9. FAB-HRMS: [M+H]⁺ calcd for C₁₆H₁₃N₄O₄ 325.0936, found 325.0924.

### REPRESENTATIVE EXAMPLE 15

### 4-(1,9-Benzyl-6,9-dihydro-6-oxo-1H-purin-8-yl)-4-hydroxy-2-oxo-but-3-enoic acid (24)

The relevant scheme is Scheme 5 shown below.

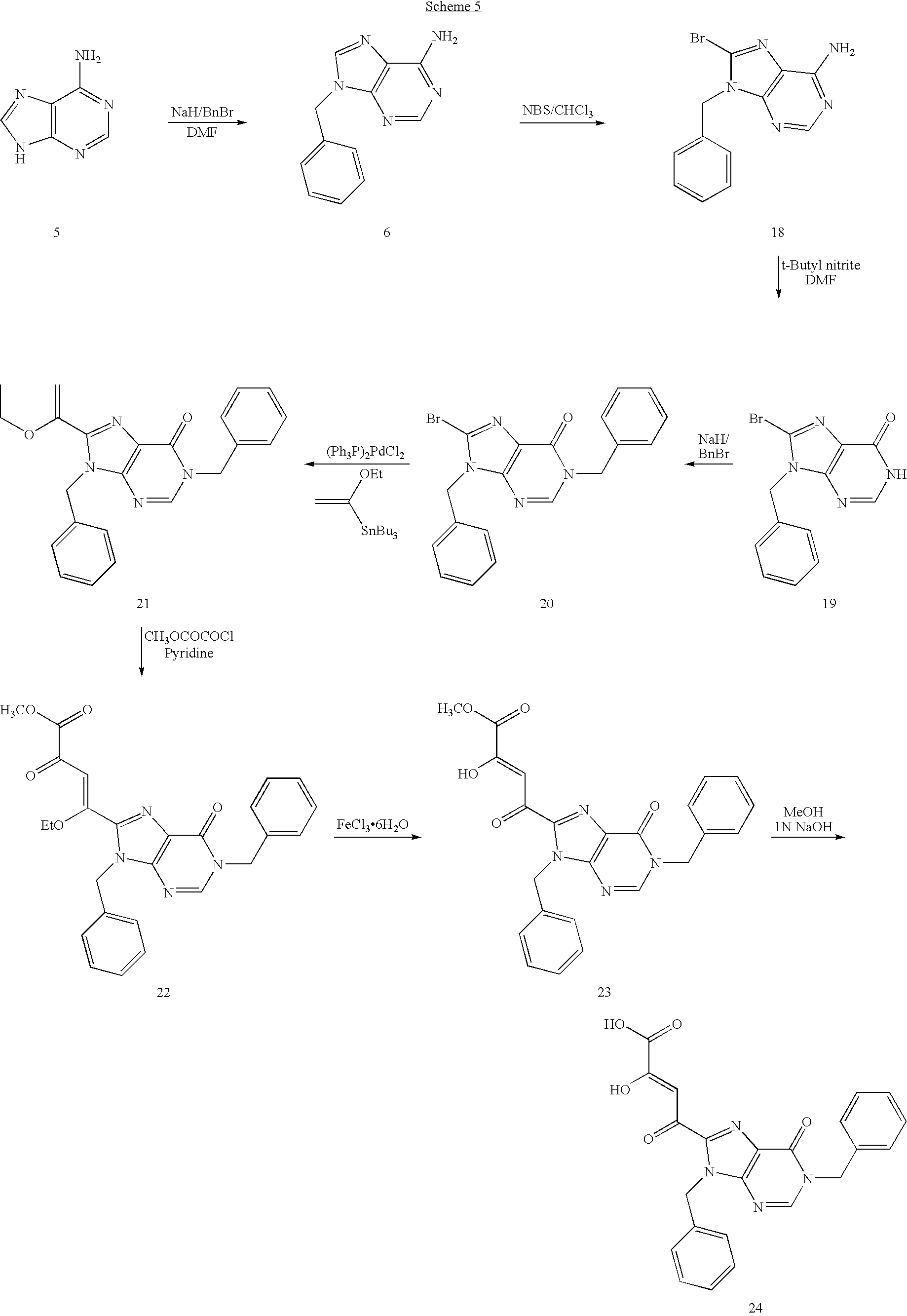

### Step 1. Described in step 1 of example 13

### Step 2: Synthesis of 9-benzyl-8-bromoadenine (18)

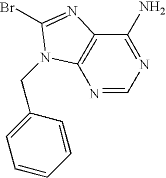

To a stirred solution of 9-benzylpurine (6) (15.0 g, 66.5 mmol) in chloroform (750 mL) was added N-bromosuccinimide (59.26 g, 332.9 mmol) and the reaction mixture was stirred under an atmosphere of nitrogen at reflux temperature for 3 h. The reaction mixture was then transferred to a separatory funnel and washed with saturated aqueous sodium sulfite (2×250 mL) followed by brine solution (2×250 mL). The chloroform fraction was dried over anhydrous sodium sulfate and concentrated to give a reddish oil, which was purified by flash chromatography on silica gel using EtOAc/hexane (4:6) for elution. Yield 9.72 g. (48%). Mp 199-201° C. ¹H NMR (CDCl₃) δ 5.39 (s, 2H, benzylic CH₂), 7.29-7.33 (m, 5H, Ar—H), 8.29 (s, 1H, purine C₂—H).

### Step 3: 9-Benzyl-6,9-dihydro-6-oxo-8-bromopurine (19)

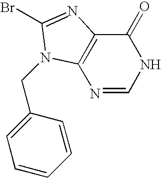

To a stirred suspension of 9-benzyl-8-bromoadenine (18) (2.60 g, 8.5 mmol) in DMF (100 mL) was added t-butyl nitrite (4.31 g, 41.8 mmol) and the reaction mixture heated under an atmosphere of nitrogen at 60-65° C. for 3 h. DMF and the excess reagent were distilled off under reduced pressure and the residue obtained triturated with EtOAc (20 mL). The yellow solid that separated was filtered off and dried under vaccum. Yield 1.41 g (54%) Mp 182-184° C. ¹H NMR (CDCl3) δ 5.39 (s, 2H, benzylic CH2), 7.21-7.42 (m, 5H, Ar—H), 8.19 (s, 1H, purine C2-H), 12.59 (s, 1H, NH).

### Step 4: 1,9-Dibenzyl-6,9-dihydro-6-oxo-8-bromopurine (20)

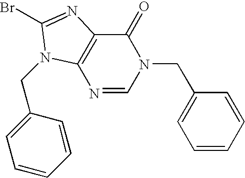

To a suspension of 9-benzyl-6,9-dihydro-6-oxo-8-bromopurine (19) (1.20 g, 3.8 mmol) in dry DMF (25 mL) was added NaH (0.113 g 4.6 mmol) followed by benzyl bromide (0.807 g, 4.6 mmol). The mixture was stirred for 15 h. at room temperature. DMF was removed under reduced pressure and the residue obtained was dissolved in EtOAc (50 mL) and washed with brine solution (2×50 mL). The organic layer was dried over anhydrous sodium sulfate and concenterated to give a yellow syrup, which was purified by column chromatography on silica gel (EtOAc: hexane, 4:6). Yield, 1.20 g (80%). Mp 161-162° C. ¹H NMR (CDCl₃) δ 5.27 (s, 2H, benzylic CH₂), 5.34 (s, 2H, benzylic CH2), 7.28-7.37 (m, 10H, Ar—H), 8.02 (s, 1H, purine C₂—H). ¹³C NMR (CDCl₃) δ 47.8, 49.3, 124.8, 126.0, 127.7, 127.7, 128.3, 128.3, 128.4, 128.9, 128.9, 129.1, 134.7, 135.8, 147.4, 149.0, 155.5, 184.1. FAB-HRMS: [M+2H] calcd. for C₁₉H₁₇BrN₄O 397.0487, found 397.0497.

### Step 5:Synthesis of 1,9-dibenzyl-6,9-dihydro-6-oxo-8-(α-ethoxyvinyl)purine (21)

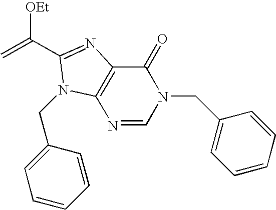

A mixture of 1,9-dibenzyl-6,9-dihydro-6-oxo-8-bromopurine (20) (1.20 g, 3.04 mmol) bis(triphenylphosphine)palladium(II)chloride (0.213 g, 0.3 mmol) and ethoxyvinyl-(tributyl)tin (2.19 g, 6.07 mmol) in dry DMF (50 mL) was heated under N₂ at 70° C. for 22 h. DMF was distilled off and the resulting residue dissolved in EtOAc (100 mL) and filtered through a pad of celite. The solvent was distilled off and the residue was purified by flash chromatography (EtOAc: hexane, 6:4). Yield 0.989 g (88%). Mp 167-168° C. ¹H NMR (CDCl₃) δ 1.26 (t, 3H, CH₃, J=7.5 Hz), 3.86 (q, 2H, CH₂, J=7 Hz), 4.46 (d, 1H, CH, J=2.5 Hz), 5.26 (s, 2H, benzylic CH₂), 5.32 (d, 1H, CH₂J=3 Hz), 5.60 (s, 2H, benzylic CH₂), 7.10-7.37 (m, 10H, Ar—H), 7.99(s, 1H, purine C₂—H). ¹³C NMR (CDCl₃) δ 14.1, 47.8, 49.1, 63.8, 90.2, 123.5, 126.6, 126.6, 127.5, 127.5, 128.2, 128.7, 128.7, 128.9, 129.1, 129.1, 136.2, 136.7, 146.2, 146.9, 148.9, 151.9, and 156.6. FAB-HRMS: [M+H]⁺ calcd. for C₂₃H₂₃N₄O₂ 387.1821, found 387.1815.

Step 6: Methyl 4-(1,9-benzyl-6,9-dihydro-6-oxo-1H-purin-8-yl)-4-ethoxy-2-oxo-but-3-enoate (22)

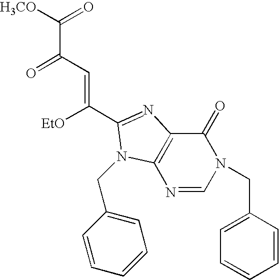

To a stirred solution of 1,9-dibenzyl-6,9-dihydro-6-oxo-8-(ethoxyvinyl)purine (21) (0.620 g, 1.6 mmol ) and pyridine (1.61 g, 19.2 mmol ) in dry chloroform (30 mL ) at 0° C. was added methyl chlorooxoacetate (1.77 mL, 19.2 mmol) in dry chloroform (10 mL) and reaction mixture was allowed to stand in the refrigerator for 48 h. The reaction mixture was washed with (2×100 mL ) water and dried over anhydrous sodium sulfate. Chloroform was distilled off to give yellow syrup from which the product was isolated by column chromatograpy (EtOAc: hexane, 4:6). Yield 0.584 g (77%). ¹H NMR (CDCl₃) δ 1.14 (t, 3H, CH₃, J=6.5 Hz), 3.66 (s, 3H, CH₃), 3.87 (q, 2H, CH₂, J=7 Hz), 5.19 (s, 2H, benzylic CH₂),5.23 (s, 2H, benzylic CH₂), 6.25 (s, 1H, olefinic CH), 7.09-7.62 (m, 10H, Ar—H), 7.98 (s, 1H, purine, C₂—H). ¹³C NMR (CDCl₃) δ 13.7, 47.3, 49.2, 52.8, 66.6, 102.6, 123.7, 127.4, 127.4, 128.0, 128.0, 128.2, 128.3, 132.1, 132.2, 135.4, 136.0, 143.8, 147.9, 148.6, 156.3, 162.0, 162.7, 181.3. FAB-HRMS: [M+H]⁺ calcd. for C₂₆H₂₅N₄O₅ 473.1824, found 473.1810.

### Step 7: Methyl 4-(1,9-benzyl-6,9-dihydro-6-oxo-1H-purin-8-yl)-2-hydroxy-4-oxo-but-3-enoate (23)

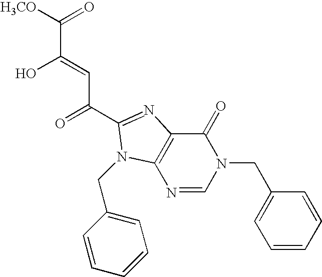

Methyl 4-(1,9-Benzyl-6-9-dihydro-6-oxo-1H-purin-8-yl)-4-ethoxy-2-oxo-but-3-enoate (22) (0.584 g, 1.2 mmole ) in CH₂Cl₂ (150 mL) was treated with FeCl₃.6H₂O (0.567 g, 2.1 mmole) and the reaction mixture was stirred at 40° C. for 3 h. The solvent was removed and the resulting residue was treated with 1 N HCl (50 mL) for 5 min, extracted with EtOAc (4×20 mL) and dried over anhydrous-sodium sulfate. The solvent was removed to give a yellowish residue which was purified by ion exchange chromatography (CH₃CN:H2O, 1:1). Yield 0.502 g. (91%). Mp 178-179° C. ¹H NMR (CDCl₃): 3.84 (s, 3H, CH₃),5.21 (s, 2H, benzylic CH₂), 5.77 (s, 2H, benzylic CH₂), 7.19-7.30 (m, 10H, Ar—H), 7.65 (s, 1H, olefinic CH), 8.07 (s, 1H, purine C2-H). ¹³C NMR (CDCl₃): δ 48.1, 49.5, 53.2, 102.5, 119.3, 124.8, 127.9, 127.9, 128.2, 128.2, 128.4, 128.7, 128.7, 128.8, 129.2, 129.2, 135.5, 135.9, 143.1, 149.4, 150.0, 156.7, 162.2, 162.2, 184.1, 185.9. FAB-HRMS: [M+H]⁺ calcd. for C₂₄H₂₁N₄O₅ 445.1511, found 445.1520.

### Step 8: Synthesis of 4-(1,9-benzyl-6,9-dihydro-6-oxo-1H-purin-8-yl)-4-hydroxy-2-oxo-but-3-enoic acid (24)

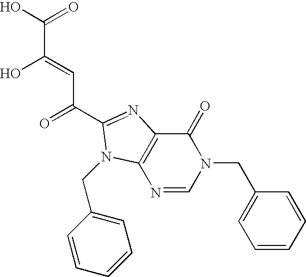

To a stirred solution of 4-(1,9-benzyl-6-9-dihydro-6-oxo-1H-purin-8-yl)-2-ethoxy-4-oxo-but-3-enoic acid methyl ester (23) (0.110 g, 0.24 mmol) in MeOH (10 mL) at 0° C. was added a solution of 1N NaOH (2 mL). The reaction mixture was allowed to stir at 0° C. for 30 min and then at room temperature for 1 h. This was followed by neutralization with 1 N HCl and the solid that separated out was filtered dried and triturated with diethyl ether to give yellow solid. Yield 91 mg (86%). Mp 167° C. (decomp.). ¹H NMR (DMSO-d₆) δ 5.27 (s, 2H, benzylic CH₂), 5.80 (s, 2H, benzylic CH₂), 7.25 (s, 1H, olefinic CH), 7.27-7.37 (m, 10H, Ar—H), 8.77 (s, 1H, purine C₂—H). 13C NMR (CDCl₃) δ 47.9, 49.4, 101.4, 123.9, 127.6, 127.6, 128.2, 128.2, 128.7, 128.9, 129.1, 137.2, 137.2, 150.4, 151.6, 156.5, 163.9, 176.0, 179.5. FAB-HRMS: [M+H]⁺ calcd for C₂₃H₁₉N₄O₅ 431.1355, found 431.1373.

We claim:

1. A method of treating a virus infection wherein the causative agent is a virus of the family flaviviridae in an infected patient comprising administering to said patient an effective amount of a compound according to the general structure of formula I (including any tautomer, regioisomer, geometric or optical isomers): 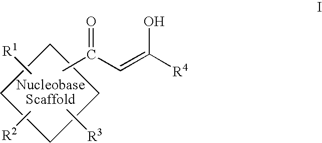
wherein the nucleobase scaffold is independently uracil, xanthine, hypoxanthine, 8-oxopurine or purine;
R¹ and R² are each independently H, C₁₋₆alkyl, C₁₋₆fluoroalkyl, unsubstituted or substituted C₅₋₆ cycloalkyl, C₁₋₆alkenyl, unsubstituted or substituted phenyl, unsubstituted or substituted benzyl, C₂₋₆alkyl phenyl which phenyl moiety may be optionally substituted, unsubstituted or substituted heteroaryl, C₁₋₆alkyl substituted with a heteroaryl group which heteroaryl group is optionally substituted, C₁₋₆alkyl S(O)R or alkyl (SO₂)R where R is alkyl, phenyl or substituted phenyl, C₁₋₆ alkyl CO₂R^{a} where R^{a} is C₁₋₆ alkyl or H, C₁₋₆ alkyl COR^{a} where R^{a} is C₁₋₆alkyl;
R³ is selected from H, C₁₋₆alkyl, halogen, hydroxyl, unsubstituted or substituted benzyl, or unsubstituted or substituted phenylthio;
R⁴ is CO₂R^{c} where each R^{c} is independently from H and C₁₋₆alkyl,
and pharmaceutically acceptable salts thereof, in combination with a pharmaceutically acceptable carrier, additive or excipient.

2. The method according to claim 1 wherein said compound has the chemical structure: 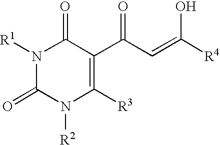
wherein R¹ and R² are each independently a benzyl group or a substituted benzyl group with 1 to 3 substituents on the aromatic ring selected from halogen, hydroxyl, methoxy, methyl, ethyl, propyl, CF₃ or a —CH₂ R^{b} group where R^{b} is a 5- or 6-membered heteroaryl group; R³ is H, C₁₋₆ alkyl, halogen, benzyl, substituted benzyl, phenylthio, or substituted phenylthio with 1 to 3 substitutents on the phenyl ring selected from halogen, hydroxyl, methoxy, methyl, ethyl, propyl, CF₃;
wherein R⁴ is CO₂R where R is selected from H and C₁₋₆ alkyl,
and pharmaceutically acceptable salts thereof.

3. The method according to claim 1 wherein said compound has the structure: 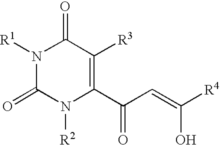
wherein R¹ and R² are each independently a benzyl group or substituted benzyl group with 1 to 3 substituents on the aromatic ring selected from halogen, hydroxyl, methyl, methoxy, ethyl, propyl, CF₃ or wherein R¹ and R² are each independently —CH₂ R^{b} where R^{b} is a 5- or 6-membered heteroaromatic ring;
R³ is selected from H, C₁₋₆ alkyl, halogen, benzyl, substituted benzyl, phenylthio, or substituted phenylthio with 1 to 3 substitutents on the phenyl ring selected from halogen, hydroxyl, methoxy, methyl, ethyl, propyl, CF₃; and
R⁴ is CO₂R where R is H and C₁₋₆ alkyl,
and pharmaceutically acceptable salts thereof.

4. The method according to claim 1 wherein said compound has the structure: 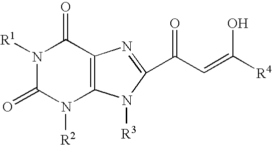
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴is CO₂R where R is selected from H and C₁₋₆ alkyl,
and pharmaceutically acceptable salts thereof.

5. The method according to claim 1 wherein said compound has the structure: 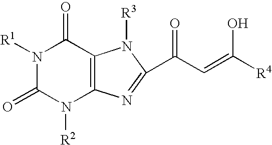
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

6. The method according to claim 1 wherein said compound has the structure: 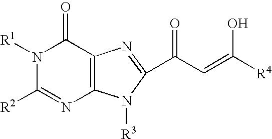
wherein R¹, R² and R³ are selected from benzyl groups or substituted benzyl groups with 1 to 3 substituents on the aromatic ring selected from halogen, hydroxyl, methoxy, methyl, ethyl, propyl, CF₃ or wherein R¹, R² and R³ are independently —CH₂R^{b} where R^{b} is a 5- or 6-membered heteroaromatic ring;
wherein R⁴ is CO₂R,
where R is selected from selected from selected from H and C₁₋₆ alkyl,
and pharmaceutically acceptable salts thereof.

7. The method according to claim 1 wherein said compound has the structure: 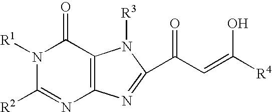
wherein R¹, R² and R³ are each independently a benzyl group or substituted benzyl groups with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from C₁₋₆ alkyl, H, sodium or other pharmaceutically acceptable salt;

8. The compound according to claim 1 wherein said compound has the structure: 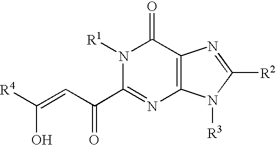
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

9. The method according to claim 1 wherein said compound has the structure: 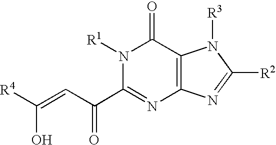
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

10. The method according to claim 1 wherein said compound has the structure: 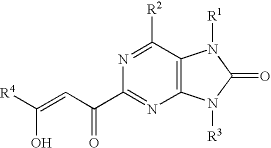
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴is CO₂R where R is selected from H and C₁₋₆ alkyl,
and pharmaceutically acceptable salts thereof.

11. The method according to claim 1 wherein said compound has the structure: 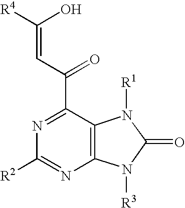
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

12. The method according to claim 1 wherein said compound has the structure: 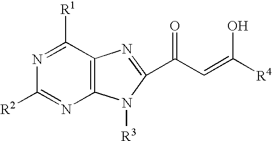
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

13. The method according to claim 1 wherein said compound has the structure: 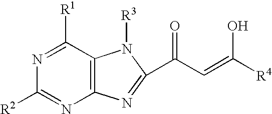
wherein R¹, R² and R³ are each indendently a benzyl group or substituted benzyl group with 1 to 3 substituents on the aromatic ring selected from halogen, hydroxyl, methoxy, methyl, ethyl, propyl, CF₃ or wherein, R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

14. The method according to claim 1 wherein said compound has the structure: 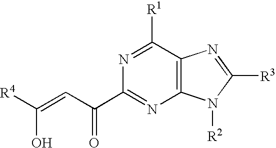
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

15. The method according to claim 1 wherein said compound has the structure: 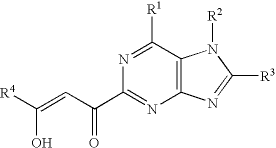
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

16. The method according to claim 1 wherein said compound has the structure: 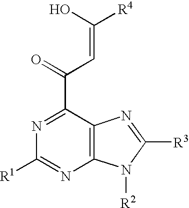
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

17. The method according to claim 1 wherein said compound has the structure: 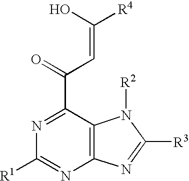
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

18. The method according to claim 1 wherein the causative agent of said virus infection is selected from the group consisting of Dengue virus, Dengue virus type 1, Dengue virus type 2, Dengue virus type 3, Dengue virus type 4, Alfuy virus, Japanese encephalitis virus, Kookaburra virus, Koutango virus, Kunjin virus, Murray Valley encephalitis virus, St. Louis encephalitis virus, Stratford virus, Usutu virus, West Nile virus, Modoc virus, Apoi virus, Rio Bravo virus, Saboya virus, the Ntaya virus, tick-born encephalitis virus, Tyuleniy virus, Uganda S virus, Yellow Fever virus, hepatitis C virus (HCV), bovine diarrhea virus, bovine diarrhea virus-2 (BVDV-2), pestivirus type 1, pestivirus type 2 and pestivirus type 3.

19. The method according to claim 18 wherein said virus is hepatitis C virus and said patient is a human.

20. A pharmaceutical composition for treating a patient infected with HIV for an HCV infection which is also present, comprising a therapeutically effective amount of a compound according to the structure: 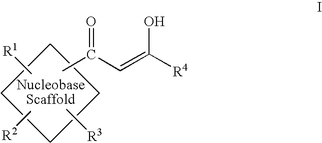
wherein the nucleobase scaffold is independently uracil, xantlline, hypoxanthine, 8-oxopurine or purine;
R¹ and R² are each independently H, C₁₋₆ alkyl, C₁₋₆ fluoroalkyl, unsubstituted or substituted C₅₋₆ cycloalkyl, C₁₋₆ alkenyl, unsubstituted or substituted phenyl, unsubstituted or substituted benzyl, C₂₋₆ alkyl phenyl which phenyl moiety may be optionally substituted, unsubstituted or substituted heteroaryl, C₁₋₆ alkyl substituted with a heteroaryl group which heteroaryl group is optionally substituted, C₁₋₆ alkyl S(O)R or alkyl (SO₂)R where R is alkyl, phenyl or substituted phenyl, C₁₋₆ alkyl CO₂R^{a} where R^{a} is C₁₋₆ alkyl or H, C₁₋₆ alkyl COR^{a′} where R^{a′} is C₁₋₆ alkyl;
R³ is selected from H, C₁₋₆ alkyl, halogen, hydroxyl, unsubstituted or substituted benzyl, or unsubstituted or substituted phenylthio;
R⁴ is CO₂R^{c} where each R^{c} is independently from H and C₁₋₆ alkyl,
and pharmaceutically acceptable salts thereof in combination with an effective amount of a second compound which is an anti-HIV agent in combination with a pharmaceutically acceptable carrier, additive or excipient.

21. The pharmaceutical composition of claim 20 wherein said composition treats said HCV infection by inhibiting HCV NS5B polymerase in the human host.

22. A pharmaceutical composition comprising an effective amount of a compound according to the structure: 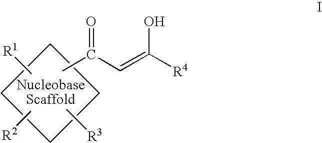
wherein the nucleobase scaffold is independently uracil, xanthine, hypoxanthine, 8-oxopurine or purine;
R¹ and R² are each independently H, C₁₋₆ alkyl, C₁₋₆ fluoroalkyl, unsubstituted or substituted C₅₋₆ cycloalkyl, C₁₋₆ alkenyl, unsubstituted or substituted phenyl, unsubstituted or substituted benzyl, C₂₋₆ alkyl phenyl which phenyl moiety may be optionally substituted, unsubstituted or substituted heteroaryl, C₁₋₆ alkyl substituted with a heteroaryl group which heteroaryl group is optionally substituted, C₁₋₆ alkyl S(O)R or alkyl (SO₂)R where R is alkyl, phenyl or substituted phenyl, C₁₋₆ alkyl CO₂R^{a} where R^{a} is C₁₋₆ alkyl or H, C₁₋₆ alkyl COR^{a′} where R^{a′} is C₁₋₆ alkyl;
R³ is selected from H, C₁₋₆ alkyl, halogen, hydroxyl, unsubstituted or substituted benzyl, or unsubstituted or substituted phenylthio;
R⁴ is CO₂R^{c} where each R^{c} is independently from H and C₁₋₆ alkyl,
and pharmaceutically acceptable salts thereof
in combination with a therapeutically effective amount of at least one additional compound selected from the group consisting of i) an anti-HIV agent, ii) an anti-infective agent other than an anti-HIV agent and iii) an immunomodulator.

23. The composition of claim 22 wherein said anti-infective agent is an antiviral agent selected from the group consisting of a protease inhibitor, a reverse transcriptase inhibitor or a combination thereof.

24. The composition of claim 23 wherein said reverse transcriptase inhibitor is a nucleoside compound.

25. The composition of claim 23 wherein said reverse transcriptase inhibitor is a non-nucleoside compound.

26. The composition of claim 20 in oral or parenteral dosage form.

27. The composition of claim 22 in oral or parenteral dosage form.

28. The composition according to claim 20 formulated for administration as an inhalation spray or a rectal suppository.

29. The composition according to claim 22 formulated for administration as an inhalation spray or a rectal suppository.

30. A method of treating an HCV infection in a patient, said method comprising administering to said patient an effective amount of a composition according to claim 20.

30. A method of treating an HCV infection in a patient, said method comprising administering to said patient an effective amount of a composition according to claim 21.

31. A method of treating an HCV infection in a patient, said method comprising administering to said patient an effective amount of a compound according to claim 22.

32. A method of reducing the likelihood of an HCV infection in a patient at risk of said infection, said method comprising administering to said patient an effective amount of a compound according to the structure: 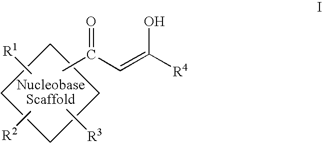
wherein the nucleobase scaffold is independently uracil, xanthine, hypoxanthine, 8-oxopurine or purine;
R¹ and R² are each independently H, C₁₋₆ alkyl, C₁₋₆ fluoroalkyl, unsubstituted or substituted C₅₋₆ cycloalkyl, C₁₋₆ alkenyl, unsubstituted or substituted phenyl, unsubstituted or substituted benzyl, C₂₋₆ alkyl phenyl which phenyl moiety may be optionally substituted, unsubstituted or substituted heteroaryl, C₁₋₆ alkyl substituted with a heteroaryl group which heteroaryl group is optionally substituted, C₁₋₆ alkyl S(O)R or alkyl (SO₂)R where R is alkyl, phenyl or substituted phenyl, C₁₋₆ alkyl CO₂R^{a} where R^{a} is C₁₋₆ alkyl or H, C₁₋₆ alkyl COR^{a′} where R^{a′} is C₁₋₆ alkyl;
R³ is selected from H, C₁₋₆ alkyl, halogen, hydroxyl, unsubstituted or substituted benzyl, or unsubstituted or substituted phenylthio;
R⁴ is CO₂R^{c} where each R^{c} is independently from H and C₁₋₆ alkyl, and pharmaceutically acceptable salts thereof.

33. The method according to claim 32 wherein said compound has the chemical structure: 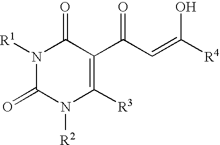
wherein R¹ and R² are each independently a benzyl group or a substituted benzyl group with 1 to 3 substituents on the aromatic ring selected from halogen, hydroxyl, methoxy, methyl, ethyl, propyl, CF₃, or a —CH₂ R^{b} group where R^{b} is a 5- or 6-membered heteroaryl group; R³ is H, C₁₋₆ alkyl, halogen, benzyl, substituted benzyl, phenylthio, or substituted phenylthio with 1 to 3 substitutents on the phenyl ring selected from halogen, hydroxyl, methoxy, methyl, ethyl, propyl, CF₃;
wherein R⁴ is CO₂R where R is selected from H and C₁₋₆ alkyl, p1 and pharmaceutically acceptable salts thereof.

34. The method according to claim 32 wherein said compound has the structure: 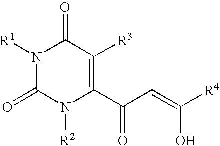
wherein R¹ and R² are each independently a benzyl group or substituted benzyl group with 1 to 3 substituents on the aromatic ring selected from halogen, hydroxyl, methyl, methoxy, ethyl, propyl, CF₃ or wherein R¹ and R² are each independently -CH₂ R^{b} where R^{b} is a 5- or 6-membered heteroaromatic ring;
R³ is selected from H, C₁₋₆ alkyl, halogen, benzyl, substituted benzyl, phenylthio, or substituted phenylthio with 1 to 3 substitutents on the phenyl ring selected from halogen, hydroxyl, methoxy, methyl, ethyl, propyl, CF₃; and
R⁴ is CO₂R where R is H and C₁₋₆ alkyl,
and pharmaceutically acceptable salts thereof.

35. The method according to claim 32 wherein said compound has the structure: 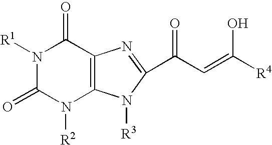
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl, p1 and pharmaceutically acceptable salts thereof.

36. The method according to claim 32 wherein said compound has the structure: 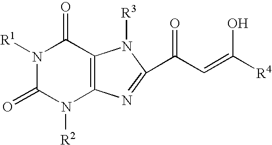
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

37. The method according to claim 32 wherein said compound has the structure: 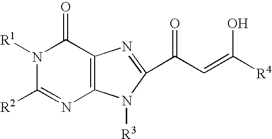
wherein R¹, R² and R³ are selected from benzyl groups or substituted benzyl groups with 1 to 3 substituents on the aromatic ring selected from halogen, hydroxyl, methoxy, methyl, ethyl, propyl, CF₃ or wherein R¹, R² and R³ are independently —CH₂R^{b} where R^{b} is a 5- or 6-membered heteroaromatic ring;
wherein R⁴ is CO₂R,
where R is selected from selected from selected from H and C₁₋₆ alkyl,
and pharmaceutically acceptable salts thereof.

38. The method according to claim 32 wherein said compound has the structure: 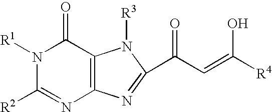
wherein R¹, R² and R³ are each independently a benzyl group or substituted benzyl groups with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from C₁₋₆ alkyl, H, sodium or other pharmaceutically acceptable salt;

39. The compound according to claim 32 wherein said compound has the structure: 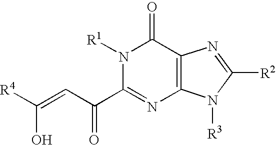
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

40. The method according to claim 32 wherein said compound has the structure: 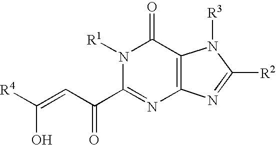
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

41. The method according to claim 32 wherein said compound has the structure: 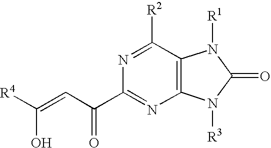
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

42. The method according to claim 32 wherein said compound has the structure: 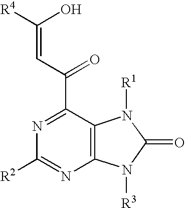
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

43. The method according to claim 32 wherein said compound has the structure: 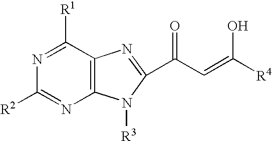
wherein R¹, R² and R³ are each independently a benzyl group or substituted benzyl group with 1 to 3'substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

44. The method according to claim 32 wherein said compound has the structure: 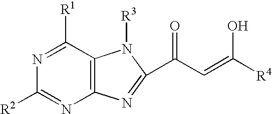
wherein R¹, R² and R³ are each indendently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

45. The method according to claim 32 wherein said compound has the structure: 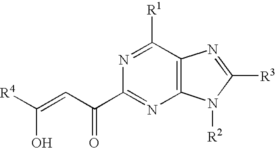
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

46. The method according to claim 32 wherein said compound has the structure: 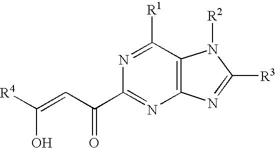
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

47. The method according to claim 32 wherein said compound has the structure: 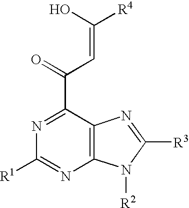
wherein R¹, R² and R³ are each independently a benzyl group or substituted benzyl group with I to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

48. The method according to claim 32 wherein said compound has the structure: 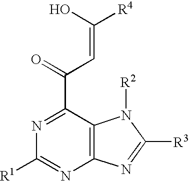
wherein R¹, R² and R³ are each independently a benzyl group or substituted benzyl group with 1 to 3 substituents on the aromatic ring selected from halogen, hydroxyl, methoxy, methyl, ethyl, propyl, CF₃ or wherein R¹, R² and R³ are each independently —CH₂ R^{b} where R^{b} is a 5- or 6-membered heteroaromatic ring;
wherein R⁴ is CO₂R where R is selected from H and C₁₋₆ alkyl,
and pharmaceutically acceptable salts thereof.

49. A method of inhibiting HCV NS5B polymerase in a subject, said method comprising administering to said subject a therapeutically effective amount of a compound according to the chemical structure: 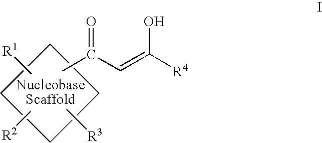
wherein the nucleobase scaffold is independently uracil, xanthine, hypoxanthine, 8-oxopurine or purine;
R¹ and R² are each independently H, C₁₋₆ alkyl, C₁₋₆ fluoroalkyl, unsubstituted or substituted C₅₋₆ cycloalkyl, C₁₋₆ alkenyl, unsubstituted or substituted phenyl, unsubstituted or substituted benzyl, C₂₋₆ alkyl phenyl which phenyl moiety may be optionally substituted, unsubstituted or substituted heteroaryl, C₁₋₆ alkyl substituted with a heteroaryl group which heteroaryl group is optionally substituted, C₁₋₆ alkyl S(O)R or alkyl (SO₂)R where R is alkyl, phenyl or substituted phenyl, C₁₋₆ alkyl CO₂R^{a} where R^{a} is C₁₋₆ alkyl or H, C 1₆ alkyl COR^{a′} where R^{a′} is C₁₋₆ alkyl;
R³ is selected from H, C₁₋₆ alkyl, halogen, hydroxyl, unsubstituted or substituted benzyl, or unsubstituted or substituted phenylthio;
R⁴ is CO₂R^{c} where each R^{c} is independently from H and C₁₋₆ alkyl,
and pharmaceutically acceptable salts thereof.

50. The method of claim 49 wherein said subject is a human.
